(19) United States
(12) Patent Application Publication
(10) Pub. No.: US 2001/0031469 A1
(43) Pub. Date: Oct. 18, 2001

(54) Methods for the detection of modified peptides, proteins and other molecules

(76) Inventor: Stefano Volinia, Ferrara (IT)

Correspondence Address: STEFANO VOLINIA, VIALE XXV APRILE 71, FERRARA, 44100, IT

(21) Appl. No.: 09/753,114

(22) Filed: Jan. 2, 2001

Related U.S. Application Data
(60) Provisional application No. 60/174,171, filed on Jan. 3, 2000.

(51) Int. Cl. C12Q 1/68

(52) U.S. Cl. 435/6; 435/7.1

(57) ABSTRACT

Method for the molecular analysis of complex samples, including biopsies from cancer and other multifactorial diseases. The method uses arrays of proteins and enzymes substrates, including peptides, antibodies, non peptide substrates and phospho-protein and acetyl-protein traps. In an embodiment tagged substrates are mass reacted in solution with the sample under investigation and then sorted onto a solid surface array by means of the relative tags. In another embodiment the substrates are immobilized onto a solid surface prior to sample analysis.

This application claims the priority of U.S. Provisional Application No. 60/174,171, Methods for the Detection of Modified Peptides, Proteins, and other Biological Molecules, filed Jan. 3, 2000, which is incorporated herein by reference.

### BACKGROUND OF THE INVENTION

1. Field of Invention

The present invention relates to the field of molecular biology and cell biology. Specifically, provided is an assay method for detection of post-translationally modified proteins and other modified biological substrates. The invention relates to methods for identifying target proteins capable of binding to and/or serving as enzymes or molecular adapters involved in biological functions. For example, the present invention distinguishes the molecular activity profiles of normal cells from those of pathological cells, or else of two different proteins or enzymes samples. The invention also relates to novel proteins or novel compounds identified using this method.

2. Description of Prior Art

In multifactorial diseases molecular heterogeneity is widespread and mutation analysis in recent years has been used as the mean to investigate the patient status. DNA sequencing and DNA microarrays are now respectively widespread or emerging technologies for molecular analysis. Often diseases of genetic origin are related to more than one gene, and the interaction with the environment has a significant impact on the outcome of the disease. These diseases are named multifactorial diseases and are still poorly understood as of their pathogenesis. Genetic background has a major influence on the manifestation of multifactorial diseases, in which severe complications may be caused through an interaction with additional factors, which may be also genetically determined. Rather than focusing on the myriad of gene mutations which can lead to altered status of a large number of proteins, we in fact aim to detect the aberrant protein status itself, which is caused by DNA mutations affecting tumor related pathways.

We describe here a new method, which can be used to integrate and/or substitute DNA analysis. We used substrate, antibodies and binding domains specific for sets of cellular proteins, in order to identify subsets of altered proteins from biopsies and other biological samples. The detection of the mutated proteins is then performed by a variety of methods, ranging from biotinylation, radioactive labeling, to immunochemistry.

This approach is of importance for the following reasons: i, it detects the net effect of a possible set of mutations, i.e. the increased activity of a protein can be the result of many different mutations, either in the same gene, or in different genes, in cis, or in trans; ii, it detects the biochemical status of a cellular compounds, thus paving the way to the use of specific drugs; iii, it is of a parallel nature, thus it is amenable of mass production; iv, it is fast, since it can be performed in few hours after the sample is obtained.

### BRIEF SUMMARY OF THE INVENTION

In order to perform the best possible therapy for a patient having a complex multifactorial disease, it is necessary to detect the proteins that have an aberrant status in the disease. Compounds, which can specifically regulate modified pathological proteins, will be then used in a directed therapy. This method allows a pharmacoproteomics approach and can also be used for small molecule screening in pharmaceutical assays. The invention is directed to methods, which enable detection of modified proteins, peptides, or other substrates and the measurement of protein or enzyme activity. Application of the invention is not limited to previously known proteins, but can also be used to identify unknown proteins or novel substrates with a functional or clinical significance. A tagged substrates array consists two separate components. The first component is a tags array, i.e. a DNA or peptide nucleic acids (PNA) array with different immobilized elements in different array locations. The second component is a set of hybrid molecules, the tagged substrates, each containing a substrate attached to a tag, i.e. DNA or PNA tag. Each tag in the tagged substrates set is complementary to at least one element in the tags arrays. Many elements in the tags arrays may not have a corresponding complementary tag in the tagged substrates set, that means the tags array might be redundant. By performing hybridization is possible to sort in a preordered fashion the tagged substrates onto the tags array. Detection of the sorted substrates it is performed by a variety of means including for example radioactive labeling, fluorescence and chemiluminescence. As a consequence, specific interactions between ligands, reacted substrates, processed molecules can be measured in parallel under the same reaction conditions. As for the other recent solid surface techniques very small amounts of sample are analyzed and processed. Among the substrates, which can be attached to a tag, we include peptides, small molecules, drugs, antibodies, binding domains. Using tagged antibodies it is possible to perform for example a proteome wide immunoprecipitation. The advantage of using tagged substrates over immobilized substrates are many and include increased stability of the substrate, improved quality control, fine substrate tuning, labeling and not least lower production costs. A tagged substrate can be kept lyophilized until use, separate from the other tagged substrates, and therefore used only for the strictly necessary time while the sample is processed. Quality of each tagged compound can be verified at any stage, and a set of tagged substrates can be reintegrated of substrates or integrated with a novel substrate at any moment, provided that the single tagged substrates are maintained as separate entities until they are processed or assembled in a pool. This also enables the use of a single universal or very few different tags arrays. Substrates can be changed, refined, or differentially labeled, at any time, without the need for designing or printing a new tag array. An important consequence is that the printing costs per array are much lower and the reproducibility higher when comparing tagged substrates arrays to immobilized substrates arrays.

DNA microarrays (Fodor et al U.S. Pat. No. 5,744,305, Lockhart et al. U.S. Pat. No. 6,040,138 ), which are entering a wide use nowadays, will give a comprehensive response on cellular RNA expression profiles, and relevant DNA mutations; but not on protein levels and activities, which ultimately constitute the cellular machinery responsible for transformation, metastasis and all other physiological or pathological changes. Protein and activity levels of many genes need to be quantitatively assessed, and this is still nowadays performed in a one-to-one protein fashion, with slow turnover and difficult comprehensive quantitative analysis. Tagged substrates arrays will enhance the experimental turnover and enlarge the population of tested proteins, effectively leading to a high throughput proteome-wide analysis. High number of internal controls will establish a robust quantitative comparison of protein levels and activities. It will be possible to study inducible complex formation, the micro-engines assembling so many cellular networks, by using a combination of tagged substrates or antibodies. It will be therefore possible to determine enzyme activity for many different enzymes in parallel. A feature of paramount importance not only for molecular diagnostics but also for pharmacogenetics and small molecule screening.

In synthesis, this novel approach allows to speed up and refine molecular analysis, improve sensitivity, and better define the enzyme activity modulation patterns at the proteome level, features that no other existing method can currently possess.

In a test for this method, more than twenty different protein binding modules have been used, including SH2, PTB, 14-3-3, bromodomains and WW domains, to detect multiple phosphorylation and acetylation events and to screen biopsies from cancer patients, using head and neck tumor and colon cancer as model neoplasia. In combination with a range of antibodies we have detected aberrant phosphoproteins in all patients and demonstrated a high correlation between the markers and metastatic progression. The advantages are manifolds: (1) it is based on the intrinsic specificity of the binding domain, natural molecules with high affinity and inherent sequence recognition; (2) its high avidity for the activated ligand, but not for the quiescent form, allows displacement of pre-existing interactions, and therefore grants access to already engaged binding sites, eventually not available to an antibody; (3) it supports correlation between recognized sites on the receptor and their biological activity; i.e. when p85 anchors to a receptor, the mechanisms leading to Akt activation are on; (4) it is not target specific, but anchor specific; in fact rather than detecting activation of a single receptor species, like a phospho- or acetyl-antibody, a binding domain recognizes a common site, present on a range of transducing molecules, where it provokes a similar molecular response, and thus prevents the need for a range of different antibodies.

The system has also some advantages when compared to genotyping (analysis of nucleotide sequence in oncogenes and tumour suppressor genes): (1) while for DNA mutations, it has to be demonstrated effective transcription and translation, and cellular influence, the identification of specific molecular patterns in biopsies from patients reveals a proteomic mutation “de facto” present in the cell and with an appropriate biochemical function; (2) activated enzymes and adapters can be themselves a pleiotropic effect, and represent the final result of different gene mutations, like i.e. a constitutive active kinase, or an inactive phosphatase (Tonks, “Introduction: Protein tyrosine phosphatases” Seminars in Cell Biology, vol. 4, pp. 373-377, 1993). This is often the case with naturally occurring cancer where mutations are distributed on different chromosomes and in a variety of loci.

In synthesis, in recent years most molecular oncology studies were conducted on nucleic acids, and thus addressed to the fine genetic dissection of the neoplastic pathologies. Here we demonstrate a system to investigate modified protein complexes in biological samples in order to classify and characterize multifactorial diseases at the molecular level. The metastatic potential of tumors can be evaluated by the quantitative detection of activated phosphoprotein complexes involved in signal transduction, such as p85 and SHC (Harrison-Findik D, Susa M, Varticovski L Association of phosphatidylinositol 3-kinase with SHC in chronic myelogeneous leukemia cells. Oncogene 1995 10:1385-91.), Fyn, Pin1, 14-3-3. The assay may employ a binding molecule, which binds to phosphotyrosines (pTyr) (Fantl W. J., Escobedo J. A., Martin G. A., Turck C. W., del Rosario M., McCormick F. and Williams L. T. “Distinct Phosphotyrosines on a Growth Factor Receptor Bind to Specific Molecules That Mediate Different Signaling Pathways” Cell, vol. 69, No. 3:413-423, 1 May 1992), phosphoserines (pSer) or phosphothreonines (pThr) in a sequence specific manner. Such binding molecule may be an SH2 or PTB domain, a WW or 14-3-3 domain or an antibody to a specific phosphotyrosine phosphoepitope. Other domains capable of detecting activated complexes in cancer are bromodomains of p300, pCAF and GG1, which can detect acetylation of lysines in modified protein complexes.

It is described an assay for the detection of modified proteins present in biological samples, such as, for example, metastatic cancer cells. A number of phosphopeptides capable of disrupting the identified complexes have been designed, to interfere in the pathological pathways leading to cell proliferation and movement and extracellular matrix invasion. These biological properties can be exploited to detect for the presence of metastatic prone tumor cells and to prevent metastatic spreading, and also to detect pre-cancerous states or unidentified cancers with an abnormal endocrine activity.

The following new findings are also described: (1) In metastatic cancer cells, but not or to a very limited extent in non-metastatic cells nor in normal cells, Shc proteins are present in a complex which bind in a phosphotyrosine dependent fashion to the SH2 domains of p85 subunit of PI 3-kinase; (2) Shc protein bind to the p85 complex in human tumors via the PTB or the SH2 domains. Peptides from the prototypical docking sites on Shc SH2 and PTB domains were designed and synthesized. These peptides are capable of interfering in the cellular pathways leading to metastasis. A double phosphopeptide was designed and synthesized with spacers for targeting the complex p85/SHC; (3) another marker which can be used to detect modified protein complexes is the Fyn-SH2 in combination with anti-phosphotyrosine antibody; (4) other markers, which can be used in order to diagnose cancer status and described in this invention, are Pin1 in combination with anti-phosphotyrosine detection, Pin1 in combination with anti-phosphothreonine antibody, and Pin1 in combination with anti-phosphoserine antibody. These markers can detect a cancer state and a metastatic state; (5) 14-3-3 bound phosphothreonine proteins are specific for cancer and metastatic cells and constitute an additional marker.

### BRIEF DESCRIPTION OF THE DRAWINGS

FIG. 1. is illustrative of examples of tagged substrates.

FIG. 2 is illustrative of examples of tagged antibodies for Rb phosphorylation status analysis.

FIG. 3 is illustrative of a single DNA/PNA tag array element (atcgtcgatgctcaa) and an hybridized tagged substrate (EAIYAAPFAK) for a specific tyrosine kinase (i.e. Abl), sorted on the respective array location (X,Y) by the complementary DNA/PNA molecule (ttgagcatcgacgat) attached onto the surface.

FIG. 4 is illustrative of a calibration with overexpression of single modifier enzymes as purified recombinant proteins and/or into a cell system. Six different tagged substrates, from the mix comprising thousands of different tagged substrates are phosphorylated by the purified enzyme under investigation, i.e. Abl tyrosine kinase.

FIG. 5 is illustrative of a read out of a non-pathological quiescent cell: at each location in the tag array it has been hybridized a single specific DNA/PNA tag coupled substrate that corresponds to a different cellular kinase and its activity. Signal intensity at the location correlates with the amount of processed substrate.

FIG. 6 is illustrative of a read out of a pathological cell where is present an activated enzyme with the substrate specificities of that in FIG. 4: at each location in the matrix has hybridized a single DNA/PNA tag and coupled substrate, that corresponds to one or more cellular kinases and its activities. Intensity of the location correlates with the amount of processed substrate. Comparison with the calibration patterns allows the identification of the protein kinase of FIG. 4 (i.e. Abl) as the activated tirosine kinase in a cellular background comprising other active kinases.

FIG. 7 is illustrative of the identification of the differences between the quiescent cell (in FIG. 6) and the pathological cell (in FIG. 6) by subtraction of the 5 matrix from the 6 matrix. FIG. 8 is illustrative of how, by further subtracting the calibration pattern relative to the Abl enzyme, it is possible to detect additionally activated enzymes, probably downstream activated from Abl.

FIG. 9 is illustrative of a protein array, with glass-bound immobilized acetyllysine-containing peptides and negative controls. In this case the substrates are not sorted but immobilized previous to the sample analysis.

FIG. 10 is illustrative of a tagged substrates array, with sorted tagged peptides after a protein kinase reaction.

FIG. 11 is illustrative of Fyn SH2 affinity purified tumor and control samples in combination with anti-pTyr immuno-detection.

FIG. 12 is also illustrative of Fyn SH2 affinity purified tumor and control samples in combination with anti-pTyr immuno-detection.

FIG. 13 is illustrative of Fyn SH2 affinity purified tumor and control samples in combination with anti-p85 immuno-detection.

FIG. 14 is illustrative of Grb2 affinity purified tumor and control samples in combination with anti-pTyr immuno-detection.

FIG. 15 is illustrative of p85 affinity purified tumor and control samples in combination with anti-SHC immuno-detection.

FIG. 16 is illustrative of Shc PTB affinity purified tumor and control samples in combination with anti-p85 immuno-detection.

FIG. 17 is illustrative of Shc SH2 affinity purified tumor and control samples in combination with anti-p85 immuno-detection.

FIG. 18 is illustrative of Pin1 affinity purified tumor and control samples in combination with anti-pThr immuno-detection.

FIG. 19 is illustrative of Pin1 affinity purified tumor and control samples in combination with anti-pTyr immuno-detection.

FIG. 20 is illustrative of 14-3-3 affinity purified tumor and control samples in combination with anti-pThr immuno-detection.

FIG. 21 Flow chart showing the procedure used to perform analysis of the combined data obtained by using the marker array of protein binding domains and antibodies.

### DETAILED DESCRIPTION OF THE INVENTION

The examples which follow show that substrates arrays, in combination with suitable antibodies or other detection systems, can be used to detect abnormal proteins in protein samples and biopsies from patients affected by cancer and other multifactorial diseases and consequently to precisely classify different molecular profiles.

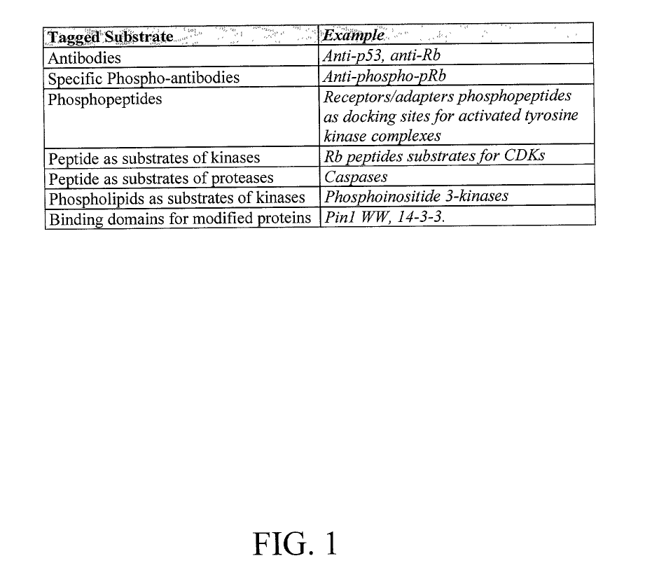

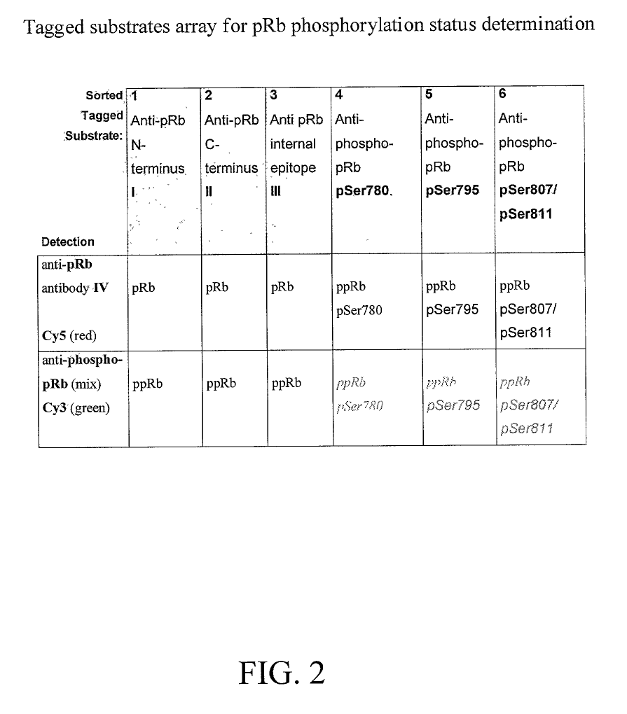

The term “modified protein complex” (MPC), as used herein, means a complex formed by a variety of proteins and including phosphotyrosine, phosphothreonine, phosphoserine, acetyllysine and other modified proteins, peptide and biological molecules. Pathologies, which may be analyzed in accordance with the present invention, include metastatic cancers and non-metastatic cancers, which can be differentiated at the molecular level. Although the scope of the present invention is not to be limited to any particular theoretical reasoning, applicants have found that, unlike normal samples, in cancers p85/SHC and other here described MPCs are associated with oncogenic transformation and metastasis. FIG. 1 describes some of the substrates, which can be used in tagged substrates assays. Different detection protocols can be evaluated as for their sensitivity and specificity. Total cellular proteins can be biotinylated. In addition cells can be fractionated prior to protein labeling, to differentiate between cytoplasmic and nuclear proteins. This procedure enables to perform indirect fluorescence detection by using for example streptavidine coupled to Cy3 or Cy5. Alternatively an antibody mix including fluorescent antibodies against all the expected captured antigens can be used in sandwich hybridization, as it is described in FIG. 2 for retinoblastoma. In order to ensure the identities of the captured proteins, which normally is enabled in western blotting by size determination, here it is necessary to array, or differentially tag and sort, different antibodies to different epitopes of the same protein. I.e. willing to detect Rb levels, at least three different anti-Rb antibodies, will be attached or sorted onto the arrays. Additionally different anti-phospho-Rb antibodies are also included in the array or tagged substrates pool, to assess Rb phosphorylation. Total cellular phosphoproteins can be visualized with ³³P labeling. ³³P labeling is probably not possible using current phosphorimagers (resolution of, at the best, 25 micron), when more than about 5000 spots/array are to be detected, unless photographic emulsion are used coupled with microscopy, but it is likely to be possible in the upcoming phosphorimagers. The same task can also be performed in sandwich hybridization with anti-pTyr, anti-pSer or anti-pThr fluorescent antibodies. It might not be possible also a general use of secondary antibodies, because of their cross reactivity with the arrayed or sorted antibodies, unless different species antibodies (e.g. chicken, rabbit, goat, sheep, rat or mouse) are used respectively for arrays and for detection. On the other hands usage of four different fluorophores-coupled to anti pTyr, anti pSer, anti pThr and anti acetyl lysine antibodies allows simultaneous detection of four different modification events in arrayed or sorted elements, at high density in a microarrays scanner with a current resolution of down to 5 micron. As detailed above domains, peptides and non-peptide substrates are to be used as counterparts to antibodies as arrayed or sorted elements. Protein binding domains almost invariably are promiscuous in their binding specificities, and their usage in protein microarrays has to be taken with caution; nevertheless it needs to be envisaged and tested, since modular protein interactions are at the very basis of control of cell functions. Domains might not only be used as immobilized or sorted baits, but also for detection in an array overlay assay, where one or more fluorescently labeled domains are used to sandwich probe molecules arrayed or sorted onto an array. A strong feature of protein arrays and tagged substrates arrays is its potential for assessing activity of a range of different enzymes. A few thousand kinases are present in the human cells, and presumably hundreds of them are present in each single cell. Their activation has been shown to be responsible for many cell cycle events. Their activity does often not correlate well with protein level, but more with post-translational modifications, or complex formation. The use of ordered arrayed or tagged peptides as substrate of a total cell kinase reaction could reveal the identity of the activated kinases present in the tumor biopsy, when compared to the normal tissue. Detection is performed by using fluorescent anti-pTyr, anti-pSer and anti-pThr antibodies as explained above. Other enzymes, which are often involved in oncogenesis, are phosphatases and proteases. Both activities can be evaluated by using synthetic peptides. For phosphatases phosphopeptides will be used and their dephosphorylation measured by using fluorescent anti-pTyr, anti-pSer and anti-pThr antibodies. For proteases (e.g. caspases) synthetic peptides containing a biotin or other modification at the C-terminus it is used. Cleavage of the peptides removes the C-terminal modification and thus a specific detection (for example fluorescent-coupled streptavidine) will quantify the amount of unprocessed peptides.

The following is a general description of the method including the preferred method. It is not intended to disclose every mode to practice the invention, and substitutions and modifications to the steps described herein may be made without departing from the scope of the invention. This invention will be better understood by reference to the following examples, which are included here for purposes of exemplification only and are not to be construed as limitations.

### EXAMPLE 1

### Protein Microarrays: Binding Domains and Modified Peptides Attached to a Solid Support for Parallel Analysis of Biopsies

Materials and Methods

Cloning and Expression of GST Fusion Proteins. A range of binding domains or whole adapter proteins have been used, such as GST-Vav, GST-Fps-SH2, GST-PTB Shc, GST-SH2 Shc, GST-Grb2, GST-SH2 Rlk, GST-Csk SH2, GST-PLC-gamma C-ter SH2, GST-PLC-gamma N-ter SH2, GST-Fyn SH2, GST-Zap70 SH2, GST-Abl SH2, GST-Syk N-ter SH2, GST-Syk C-ter SH2, GST-p85 full length. Pin1 cDNA was amplified by using RT-PCR and proofreading Pfu DNA polymerase, fully sequenced and cloned into BamHI and EcoRI linearized pGeEX2-TK. RT-PCR was used in order to clone different bromodomains in BamHI/EcoRI cleaved pGEX2TK vector (Pharmacia), by using turbo-Pfu DNA polymerase (Stratagene) and human first strand cDNA from a range of human cell lines. Oligonucleotides used for pCAF sense CCGGGATCCAGTAAAGAGCCCAGAGACCC, antisense CCAGAATTCTCACTTGTCAATTAATCCAGC, p300 sense (BglII) GaagatctAAAAAGATTTTCAAACCAGAAGAAC, antisense (EcoRI) CGGAATTCTCATTGCATCACTGGGTCAATTTC, CG1-1 sense TGGGATCCCGCACAGACCCTATGGTGAC, CG1-1 antisense GGAATTCTCATTTCTCTTTGAGTTTTTCATCACAG. The clones were sequenced to confirm their identity. The domains are expressed as GST fusions in E.coli and used in affinity purification assays, from lysates of tumor and control samples, deriving from the dame patient. All proteins were harvested after sonication of IPTG treated bacterial pellets in lysis buffer (20 mM Tris-HCl, pH 8.0, 137 mM NaCl, 10% glycerol, 1.0% Triton X-100, 1 mM phenylmethylsulfonyl fluoride, 0.15 unit/ml aprotinin, and 20 μM leupeptin) and clarified by centrifugation at 13,000 g Oriented acetyl lysine peptide library assays. One mg of GST-bromodomain bound to 200 microliters of glutathione Sepharose was packed in a spin micro-column and washed twice with binding buffer (PBS Dulbecco, Gibco BRL) (1×PBS,, 0.5 mM MgCl2, 0.9 mM CaCl2) with 0.1% Tween 20. Two and a half milligrams of acetyl-lysine peptide library (MAXXXX-AcK-XXXXAKKK) in 500 microliters of binding buffer was applied onto the column, and peptide was absorbed at RT for 10 minutes with gentle agitation. The binding buffer was removed by centrifugation at 2000 rpm for 120 seconds. Three subsequent washing steps were performed with 500 microliters of ice-cold binding buffer and quick centrifugation as above. Elution was performed with 250 microliters of binding buffer containing 20 mM N-acetylhistamine, in binding buffer, incubating the column for 10 minutes at RT, and centrifugation. Eluate was monitored on a HP maldi tof mass spectrometer. The collected peptide was lyophilized and sequenced. Preparative purification use acetyl-histamine to specifically elute bromodomain bound peptide library.

Purification of proteins from human tumour samples. Lysates were produced with the microdismembrator from frozen samples and the frozen powder was resuspended with 2.0 mls of standard lysis buffer (137 mM NaCl, 20 mM Tris-HCl (pH 7.4), 10% glycerol, 1% NP-40, 150.mu.g/ml aprotinin and leupeptin, pepstatin, 2 mM EDTA, sodium orthovanadate, 1 mM NaF) and incubated at 4.degree. Celsius with constant rocking for 15.minutes. Lysates were cleared by centrifugation at 12,000.times.g for 5 minutes at 4.degree. Celsius.

Protein microarrays spotting and detection. Proteins (5 nanoliters) were spotted and covalently attached onto 3D-link activated slides (Surmodics Inc.) by using a robotic arrayer and the standard protocol. Slides were blocked for 1 hour at room temperature in 5% BSA and hybridization buffer (20 mM Tris-HCl pH 7.8, 150 mM NaCl 0.02% Tween 20). Detection of the protein microarray bound material was performed with anti-acetyl lysine polyclonal antibody (NEB) at 37 C for 15 minutes, followed by secondary anti-rabbit FITC-conjugated. After three washes in hybridization buffer for 1 minute each, the slides were dried and scanned using a Molecular Imager FX (BioRad) or a fluorescence microscope.

Results and Discussion

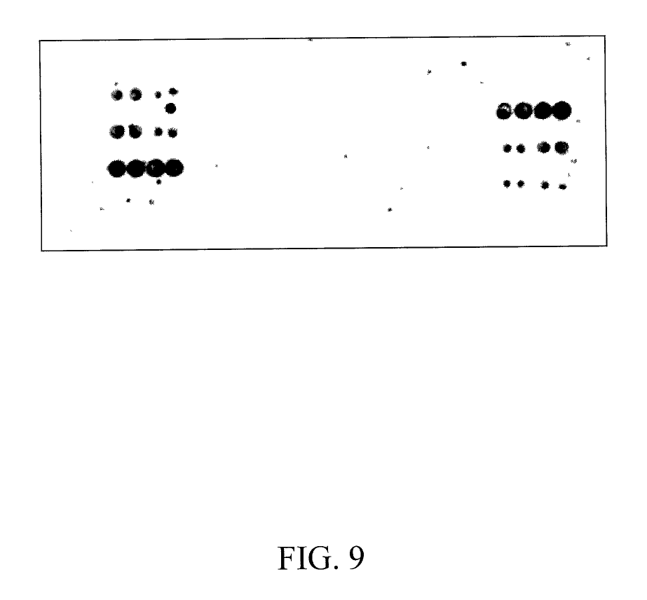

A protein microarray, with glass-bound protein-binding domains and acetyllysine-containing peptides. Protein from cell lysates were absorbed onto the surface-bound proteins for 1 hour at room temperature and washed. Anti-acetyllysine antibody was absorbed for 15 minutes on the top of the slide (FIG. 9). Detection was obtained with a Molecular Imager FX in fluorescence after binding a secondary FITC-conjugated anti-rabbit antibody.

### EXAMPLE 2

### Multiplex Modifier Enzymes Activity from a Parallel Assay in Solution and Sorted on Solid Phase: A Cell Wide Enzyme Assay

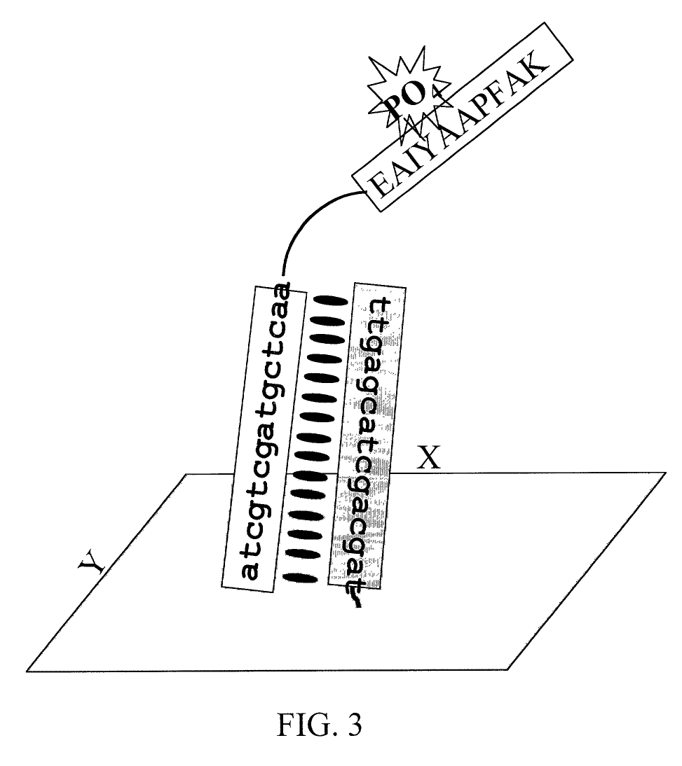

This assay allows the investigation of the activity of the modifier enzymes present in complex biological samples, such as a cell lysate or a tissue extract. It is a global assay that enables the determination of each and all the modifier enzymes present in a sample at the proteome level. It uses a peptide or any other specific substrate for each modifier enzyme under investigation, be it of known or unknown substrate specificity, as it is explained in the following description. Substrates can be of the following types: real known targets for the enzyme under study, consensus targets (even artificial and not existing in nature), putative targets (existing in nature, but not confirmed experimentally), randomly designed targets. Each substrate is univocally coupled, covalently or non-covalently, to a different DNA/PNA tag of known sequence (FIG. 3). More than one tag can be used for each substrate in order to increase the test's robustness. The DNA/PNA tag constitutes the mechanism by which the substrates are sorted, by hybridization onto an ordered matrix of tag-complementary solid surface-bound DNA/PNA molecules, after the enzyme reaction is completed.

A mix comprising a high number of different tagged substrates, i.e. a thousand or multiples of thousand, in a suitable buffer in solution is applied to the sample under investigation, which could be a cell lysate or any other biological sample. A suitable labeling reagent might be added to the reaction, such as a radioisotope in order to follow a biochemical reaction. Specific inhibitors, i.e. kinase, protease, phosphatase inhibitors, or co-factors, such as magnesium, manganese, or calcium ions, might also be added to the reaction inhibitors in order to evaluate a particular subset of enzyme reactions. The tags might be modified chemically in order not to be themselves substrates of modifier enzymes under investigation. Upon completion the reaction is stopped, chemically or physically, and the tagged substrates are possibly purified using an affinity column for the tags in order to separate them from the biological sample in study. Each tagged substrate is now sorted by hybridization, under appropriate conditions related to the DNA/PNA tags, onto a DNA/PNA tag array slide, which was previously prepared by using an ordered matrix comprising the complementary DNA/PNA to each tag of the tagged substrates (FIG. 3). Finally the sorted modified substrates are analysed, for example by Phosphorimager, if radiolabeled, or by fluorescence scanning, if using a fluorescence based detection system and a computer scan is performed in order to assign the activity value to each different enzyme. The program averages the different measures from different sorted substrates of the same activity and then prints a read out, excluding statistically non significative measurements, and highlighting the abnormal values, corresponding to deregulated enzymes, when compared to a standard.

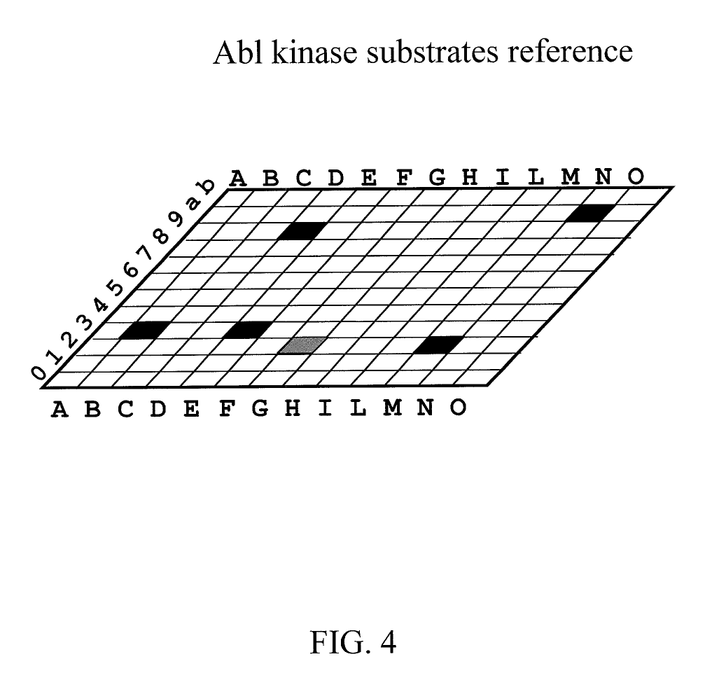

Proteome wide assignment of substrates is performed, by the user or by the provider, by using isolated purified recombinant enzymes and cellular extracts with over-expressed enzymes, in order to unequivocally assign each substrate, or substrate subset, to a modifier enzyme, i.e. the Abl tyrosine protein kinase (FIG. 4). Different intensities resulting from different locations on the matrix and corresponding to different substrates, indicate the enzymes substrate propensity. A substrate with higher affinity to an enzyme or protein will give a stronger signal and vice versa, or the reverse when the reaction is catabolic, like for example in the case of proteases or phosphatases. In the latter cases the substrate is negatively affected by the enzyme activity, and it needs to be labeled a priori in order to be visualized in a negative fashion for the processed form. Users can synthesize and add custom tagged substrates to the system, since a number of tags is kept unallocated from the producer to this purpose, and the corresponding extra complementary tags are arrayed on the solid surface sorting matrix. Particularly unstable tagged substrates are kept under optimal condition until are used in the reaction assay.

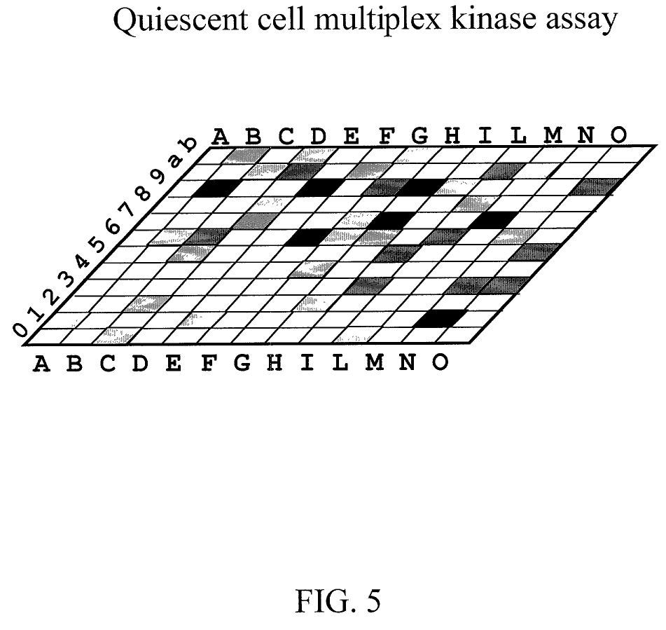

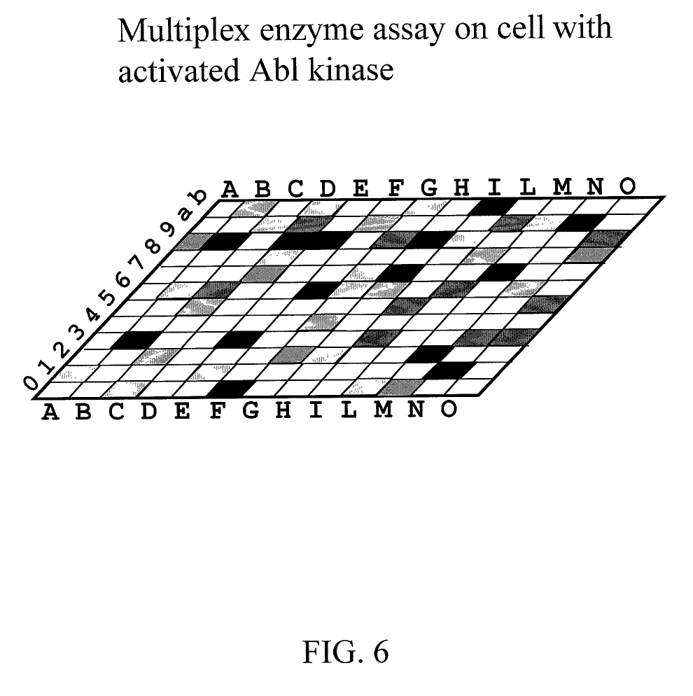

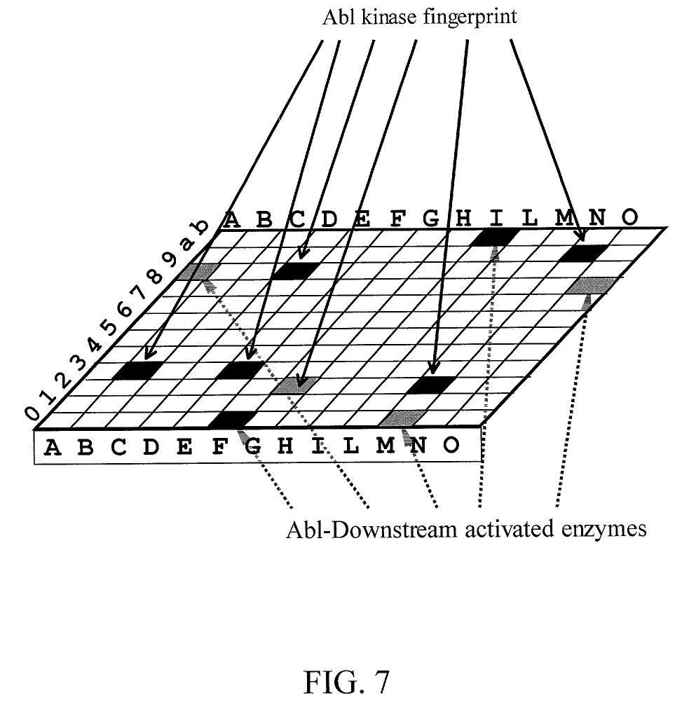

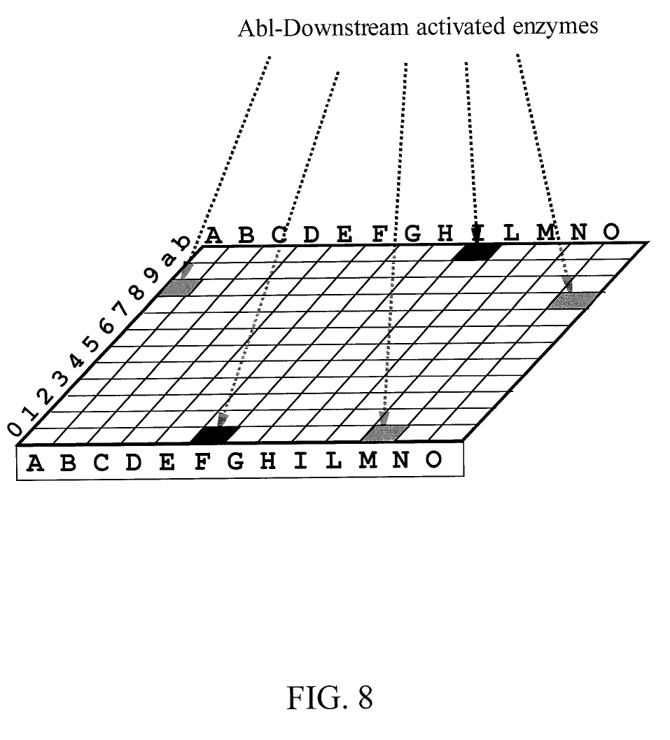

In the case of a complex sample, say a quiescent cell lysate, the results of an experiment are exemplified in FIG. 5, where it is shown the results of a cell wide kinase assay. Analyzing a cell that, for example, contains an activated Abl oncogene the resulting pattern is that of FIG. 6. By subtracting the pattern of FIG. 5 from that of FIG. 6, it is possible to obtain the fingerprint corresponding to the substrates for the kinases, which are differentially activated or expressed in the two cell samples, as it is shown in FIG. 7. Furthermore by using this system it is possible to detect members in enzyme cascade. The arrows from the top of FIG. 7 indicate the Abl substrates, while the arrows from the bottom of FIG. 7 indicate differentially activated or expressed kinases which are different from Abl, but acting possibly downstream of it.

Discussion

This method has a major advantage over expression profiling using DNA/RNA/PNA arrays, in fact the activity of kinases and phosphatases and other modifier enzymes can not be at all correlated with the abundance of their transcripts, since very frequently they are regulated by post-translational modifications, i.e. phosphorylation. Enzymes such these are in fact generally inducible/allosteric, i.e. their activity does not correlate with the molar concentration and is instead regulated heavily by post-translational events. For this reason it is not possible to rely on the expression detection systems nowadays being developed on DNA/PNA chip. Only direct measurement of the enzyme activity can assess the enzyme cellular role in a biological event, for example cancer or another disease.

An activity assay is required which can correlate enzyme with activity in a proteome wide assay. The system described here is therefore fundamental as a mean to achieve knowledge of the status of different enzymes in a complex sample.

For processive catabolic reactions such as those catalyzed by proteases, phosphatases, or other enzymes, activities can be determined if the tagged substrates contains for example a fluorescent label at the free non-tagged peptide termination. When a molecule of substrate is cleaved the fluorescent label is detached from the substrate-tagged molecule and therefore will not be detected after sorting on the array and fluorescence scan.

This method has also major advantages over ordinary immunopurification (1) it reveals parallel mass result of sample wide modifier enzymes activities; (2) the enzymes are still in complex with their molecular partner and not isolated on immunocomplexes, where their activity could be deregulated; (3) lysis can be either with detergent or with osmotic shock or any other system, as to retain as much as possible the cytosolic assembly; (4) using tagged antibodies it is possible to perform parallel immunopurifications for a large number of antigens; (5) it is possible to use modifier enzyme inhibitors, and signal transduction inhibitors to finely dissect enzyme cascades; (6) finally but not least important is possible to use non-peptide substrates, i.e. phospholipids which needs labor and time intensive analytical systems such as for example HPLC or TLC, even in the case of chemically widely different substrates, which otherwise would need different analytical systems.

This method can be used for: (1) diagnosis of pathological disorders in multifactorial diseases; (2) prognosis of pathological diseases; (3) studying the evolution of complex diseases; (4) determination of minimal residual disease; (5) determination of drug response in therapy evaluations; (6) drug discovery for alteration of enzyme mechanisms; (7) enzyme substrate specificity discovery; and (8) enzyme substrate manipulation.

Multifactorial diseases such as cancer are the result of a variety of mutations. Many of them are affecting regulatory enzymes, such as phosphatases and kinases. The net effect of many different mutations can thus have its outcome in changing the phosphorylation state of a key enzyme, such as for example receptors, signalling enzymes, transcription or tumor suppressor genes, like p53. Mutations are usually detected at the DNA level, but the phenotype can be the results of a variety of mutations in different genes. If a key protein, like for example p53, it is altered in its phosphorylation state, it can become a cause of tumorigenesis. Thus a system to identify key post-translational changes has many advantages over the traditional DNA driven mutations detection systems; i) it detects the real molecular effect, and not a mutation which might even not be expressed, if genomic, nor translated if derived from the mRNA; ii) the key change can be the result of a host of mutations in different regulative genes, some of which might be unknown in identity or function; iii) the post-translational modification is not an amino acid mutation, and thus can not be indirectly deduced by DNA or RNA assay.

This system can be used in order to study all the enzymes present in a sample in parallel, with a very high throughput. The system's advantages over current techniques is also that it gives a readout of the crosstalk within the different enzymes, i.e. it is possible to understand the relations of positive and negative feedback within divergent enzyme pathways.

The system is made robust by adopting a configuration where the same target substrate (peptide, antibody, binding domain, lipid or other molecule) is singularly linked to more than one different DNA/PNA tag, resulting in a situation were different tags of known sequence identify the same substrate, which can be sorted to different matrix positions on the solid surface. Furthermore small substrates, i.e. peptides or other molecules can be attached to the tag, in different orientations, and with different spacers, in order to avoid functional constrains. Thus the same bona fide signal can be sorted, detected, measured and statistically evaluated. By using the same substrate, coupled to different tags and at different concentrations in the substrate pool, it is also possible to perform in a single pass quantitative analysis, i.e. to calculate biochemical parameters such as Kd.

### EXAMPLE 3

### Multiplex Modifier Enzymes Activity from a Parallel Assay in Solution and Sorted on Solid Phase: A Test Enzyme Assay

Materials and Methods.

EDC/NHS combined cross-linking was performed to tag the peptide or substrate which needs to be sorted. Coupling buffer contains no Tris and no phosphate. Peptides and oligonucleotides in equimolar ratios [50 μg/ml in 10 mM sodium acetate buffer (pH 5.0)] were reacted with a 1:1 mixture of N-hydroxysuccinimide NHS and 1 -ethyl-3 -(3 -dimethylaminopropyl) carbodiimide EDC for 2 hours. The excess active groups were then blocked with 1 M ethanolamine (pH 8.5).

Peptides. Phosphotyrosine-containing and non-phosphorylated peptides were synthesized, HPLC purified and checked by mass spectroscopy. Peptides were stored under nitrogen at -80 degree. Celsius. The synthesized peptides were as following, phosphotyrosine, AEPDpYGALYE PLCgamma, SAAPpYLKTK Stat3-705, DDPSpYVNVQ SHC-317, PDHQpYYNDF SHC-239, TDDGpYMPMS IRS 1-608, GNGDpYMPMS IRS 1-628, SPGEpYVNIE IRS 1-895, KSLNpYIDLD IRS 1-1172, DLSTpYASIN IRS1-1222; non-phosphorylated, AEPDYGALYE, SAAPYLKTK, DDPSYVNVQ and PDHQYYNDF SHC, KDGATMKTF Akt, RGRSRSAPPN BAD, GEGTYGVVYK p34cdc2, GAGTPAATDEK, DGFVLTRLLE beta spectrin, NSIMKCDIDI Gamma actin, PGIADRMQKE beta actin.

Amino modified oligonucleotides used as tags. The amino modifications can be used either for slides coupling or for peptide coupling, or for coupling to any reactive substrates which needs to be sorted onto the array. Oligonucleotides are chosen with similar Tm and hybridization properties. Oligonucleotides can be substituted by any pair of complementary nucleic acids or PNAs without affecting the sensitivity or completeness of the assay. The length and sequence of the nucleic acids or PNAs is also not important as long as strands complementarity results in hibridization specificity. Here it is described a test experiment with 18 pairs of oligonucleotides. This strategy can effectively support sorting of many thousands of different substrates to even millions of different substrates, simply changing the sequence of the tag pairs used for each array location. As a rule, the tags in the tagged substrate pool are not complementary to each others, to avoid tag to tag hybridization and loss of sorted signal.

Slide Overlay. Cell samples were lysed in buffer A (10 mM Tris-HCl buffer pH 7.5, 10% glycerol, 1% Triton X-100, 150 mM NaCl), supplemented with 0.5 mM sodium orthovanadate, 50 mM NaF, 0.2 mM phenyl-methylsulfonyl fluoride, 1 ug/ml leupeptin, 0.1 TIU/ml aprotinin and 1 ug/ml pepstatin. Lysates were clarified at 15,000 g at 4 degrees Celsius for 15 minutes. Hundred micrograms of total cell lysate is reacted with the tagged substrates mix in a in vitro protein kinase reaction for 30 minutes at 37 degree. Celsius. Cell sample are HEK293 cell lysates after 5 minutes serum stimulation. After killing the reaction for 5 minutes at 90 degree. Celsius, the substrates mixture was applied onto the array in a 100 .mu.l volume for 1 hour at RT. The slide was then washed 3 times in 1× TBST and an immunostaining using anti-pTyr monoclonal antibody was subsequently performed. Blocking of nonspecific reactivity is achieved with 2% BSA, dissolved in TBST (20 mM Tris-HCl pH 7.8, 150 mM NaCl 0.02% Tween 20) (1 hr incubation at 22 degrees Celsius). Three different anti-phosphotyrosine monoclonal antibodies, 4G10, PY20 and pTyr-100, from three different companies (UBI, Santa Cruz and NEB) are used in a primary antibody mix with 2% BSA. Fluorescent Cy5 secondary anti-mouse antibody was applied at RT for 30 minutes. After triple washing in TBST, and then TBS, fluorescent complexes are detected by using a GenePix microarrays scanner.

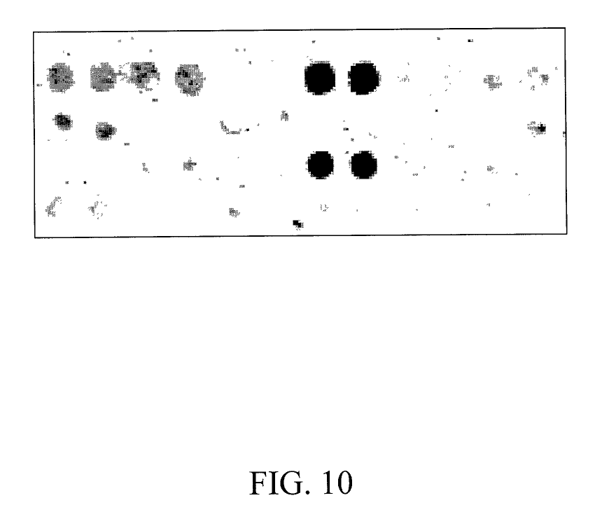

In FIG. 10 it is illustrated a hybridized tagged array where the arrayed tags are as follows: S1 NH2-GCT GAG GTC GAT GCT GAG GTC GCT, S2 NH2-CGC AAG GTA GGT GCT GTA CCC GCG, S3 NH2-GCT GTG GTC GTT GCT GCG GTC GTA, S4 NH2-CGC AGG GTT GGT GCA GTA CGC CCA, S5 NH2-GTT GAG GTC GAT GAT GAG ATC GCA, S6 NH2-CGC AAG GTA GGT GCT GTA CGC GCT, S7 NH2-GGT GTG TTC GTT GCT GAG GTC GTC, S8 NH2-CGC ATG GTT GTT GCA GTA CAC CCG, S9 NH2-ACT GAG GTC GAT CCT GAG GTC GCT, S10 NH2-CGC TTG GTA GGT GCT GTA CAC GCA, S11 NH2-GCT GTG AAC GTT GCT GCG GTC GTA, S12 NH2-CGC AGG GTT GGT GGT GTA CGC CCA, S13 NH2-GAT GAG GTC GAT GCT GAG ATC GCA, S14 NH2-GAC AAG GTA GGT GCT GTA CGC GCC, S15 NH2-GGT GTG TTC GCT GCT GAG GTA GTA, S16 NH2-AAC ATG GGT GTT GTA GTA CAC CGA, S17 NH2-ATT GAG GTC GAC CCT GAG GTC GCA, S18 NH2-CGC TCG GTA GGT GCA GTA CAC GCG. Each tag is arrayed in duplicate. Complementary oligonucleotides (AS1-AS18) to the S1-S18 tags were attached to peptides using EDC as as described and with the follow order: AS1 AEPDYGALYE, AS2 SAAPYLKTK, AS3 KDGATMKTF, AS4 PDHQYYNDF, AS5 GAGTPAATDEK, AS6 DDPSYVNVQ, AS7 GEGTYGVVYK, AS8 GAGTPAATDEK, AS9 DGFVLTRLLE, AS10 NSIMKCDIDI, AS 11 PGIADRMQKE, AS12 RGRSRSAPPN, AS13 GAGTPAATDEK, AS 14 DGFVLTRLLE, AS15 DDPSYVNVQ, AS16 PDHQYYNDF, AS17 NSIMKCDIDI, AS18 PGIADRMQKE. S1 to S18 tags are arrayed in FIG. 10 from the right to le left and from top to bottom, in pairs of duplicates. An inverted image is displayed. Substrates S1 and S2 are moderately well phosphorylated. Substrates S4 and S16 are strongly phosphorylated. Other substrates are not phosphorylated or only to a very low extent. A good reproducibility is shown in the figure by the paired spots.

### EXAMPLE 4

### Antibody Array: Retinoblastoma Protein and Phosphorylation Analysis

This array for the detection of Rb and of its phosphorylation status is illustrated in the table in FIG. 2, and is composed as follows. Tagged substrates elements: i) pRb analysis (3 different antibodies against different pRb epitopes); ii) phospho-pRb (ppRb) analysis (3 different phospho-antibodies against different pRb phosphorylation sites).

Detection: a fourth anti-pRb antibody, different by the three in the array (Cy5 red fluorescence). Detection: anti-phospho-pRb antibody mix (of the three arrayed phospho-antibodies) (Cy3 green fluorescence).

With this elements set it is possible to measure: i) the amount of pRb in elements 1, 2, 3, by Cy5; ii) the site specific phosphorylation of pRb in elements 4, 5, 6 by Cy5; iii) the total phosphorylation level of ppRb by using a phospho-antibody mix in 1, 2, 3 with Cy3; and eventually iv) to compare the amount of cellular pRb present in the biopsy with a standard added in known amounts to the biopsy (FITC channel, not displayed in the figure). Elements 4,5 and 6 read with Cy3 are not reliable, since the same antibodies are used both in array and detection, and thus are written in gray. As for many of the examples in this patent application, arrays with sorted tagged substrates can be also thought of as arrays with immobilized substrates.

### EXAMPLE 5

### Domain Array: p53 Analysis

This array for the detection of p53 and of its phosphorylation status is composed as follows. Sorted tagged substrates elements:3 different immobilized antibodies against different p53 epitopes. Detection: recombinant 14-3-3 (Cy5 fluorescence). Detection: recombinant MDM2 (Cy3 fluorescence). Detection: recombinant PARP (FITC fluorescence).

In this manner it is possible to measure: i) the amount of cellular p53 in the tumor capable of binding 14-3-3, ii), the amount of cellular p53 in the tumor capable of binding MDM2, iii) the amount of cellular p53 in the tumor capable of binding PARP.

Control: using a fourth different fluorophore (eg Texas Red) coupled to an anti-p53 antibody, better if different from those used in the microarrays, it is possible to measure the level of tumor p53 captured onto the microarray.

### EXAMPLE 6

### Peptide Array: Protein Kinase Assay

Array elements: 100 different non phosphorylated peptides, corresponding to known tyrosine and serine/threonine kinase substrates are arrayed (in the case of a peptide array, 12 immobilized peptides, spotted at 3 different concentrations, per each peptide, or else in the case of a tagged substrates 12 replicates of 3 different immobilized tags, per each substrate). Detection: anti-pTyr monoclonal antibodies (mix) (Cy5 red fluorescence). Detection: anti-pSer antibodies (Cy3 green fluorescence). Detection: anti-pThr antibodies (FITC false blue fluorescence). It is possible to detect phosphorylation of peptides and to measure the relative phosphorylation levels, thus inferring kinases activities. 3Dlink activated slides or similar activated slides are used to couple the samples either directly or as tagged substrates onto the glass surface at defined coordinates by a DNA microarrayer. In the case of tagged substrates the complementary tags are coupled onto the array.

Cy5 conjugation of Antibodies. Sodium azide is completely removed from any antibody: it reacts with the Cy5 and prevent conjugation. The antibody is dialyzed against Reaction Buffer (500 mM carbonate, pH 9.5). Cy5 in anhydrous DMSO is prepared immediately before use, at a concentration of 10 mg/ml. For the optimal ratio of 5:1, 40 μg Cy5 are added per mg of antibody, incubated and rotated at room temperature for 1 hour. The unreacted Cy5 is removed by gel filtration or dialysis into Storage Buffer (10 mM Tris, 150 mM NaCl, pHix, pH 8.2.

Biotinylation Procedure. Amine-reactive reagents react with non-protonated aliphatic amine groups, including the amine terminus of proteins and the E-amino group of lysines. The amino group has a pK a of around 10.5; in order to maintain this amine group in the non-protonated form, the conjugation must take place in a buffer with slightly basic pH. It is important to avoid buffers that contain primary amines, such as Tris, as these will compete for conjugation with the amine-reactive compound. NHS biotin is dissolved in anhydrous dimethylformamide (DMF) or dimethylsulfoxide (DMSO). Labeling of the protein or peptide amino terminus can be achieved using a buffer closer to neutral pH, pH, as the pK a of the terminal amine is lower than that of the lysine amino group. 1.5 M Hydroxylamine, pH 8.5, is used to terminate the reaction and to remove weakly bound probes. A typical labeled protein can be easily separated from free dye using a gel filtration column, such as Sephadex G-25 or equivalent, equilibrated with the buffer of your choice. For much smaller or larger proteins, other gel filtration columns may be more appropriate. For short peptides reverse phase chromatography is required.

### EXAMPLE 7

### Fyn SH2 Distinguishes pTyr Containing Modified Protein Complexes in Cancer

Materials and Methods

Expression of GST-Fyn Fusion Protein. The cDNA for the SH2 domain of the Fyn tyrosine kinase was isolated using polymerase chain reaction (PCR), cloned into the BamHI-EcoRI sites of the bacterial expression plasmid pGEX-2T (GST-FynSH2) and expressed in Escherichia coli as glutathione S-transferase (GST) fusion proteins. Protein was harvested by lysis in lysis buffer (20 mM Tris-HCl, pH 8.0, 137 mM NaCl, 10% glycerol, 1.0% Triton X-100, 1 mM phenylmethylsulfonyl fluoride, 0.15 unit/ml aprotinin, and 20 μM leupeptin) and clarified by centrifugation at 13,000 g.

In vitro association experiments. Human freshly obtained or frozen tumor and normal samples cells (approximately 2.times.10.sup.6 cells/point) were lysed in buffer A (10 mM Tris-HCl buffer pH 7.5, 10% glycerol, 1% Triton X-100, 150 mM NaCl, 5 mM EDTA), supplemented with 0.5 mM sodium orthovanadate, 0.2 mM phenyl-methylsulfonyl fluoride, 1 .mu.g/ml leupeptin, 0.1 TIU/ml aprotinin and 1 .mu.g/ml pepstatin. Lysates were clarified at 15,000.times.g at 4.degree. Celsius for 15 minutes and the supernatant affinity purified on Glutathione Sepharose bound GST-SH2 domain for 4 hours to O/N at 4 degrees Celsius. The phosphotyrosine protein complexes were washed three times with buffer A, once with buffer B (10 mM Tris-HCl pH 7.4, 100 mM NaCl, 1 mM EDTA. When checking for the ability of phosphopeptides to block the associations with the complex, cell lysates were pre-incubated with the phosphopeptides for 1 hour at 4.degree. Celsius prior to incubation with the immobilized recombinant GST-SH2. Following association, immobilized complexes were washed as described above. The SH2-bound complexes were eluted from Glutatione-Sepharose in boiling Laemmli buffer. Supernatants were then subjected to 8% sodium dodecyl-sulfate polyacrylamide gel electrophoresis (SDS-PAGE).

Western immunoblotting. Immunoprecipitates after the association were solubilized in boiling Laemmli buffer, separated on 8% SDS-PAGE and electro-transferred into nitrocellulose filters (Hi-bond, Amersham). Filters were then incubated with the indicated antibodies and specific binding was detected by the enhanced chemiluminescence system (ECL.TM., Amersham).

Results and Discussion

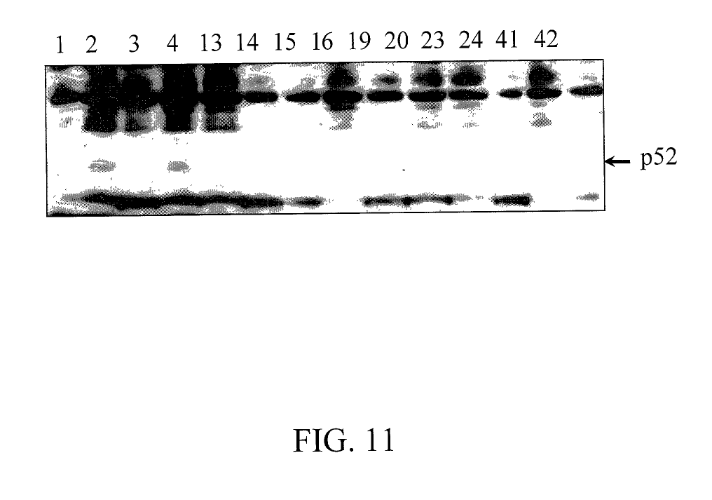

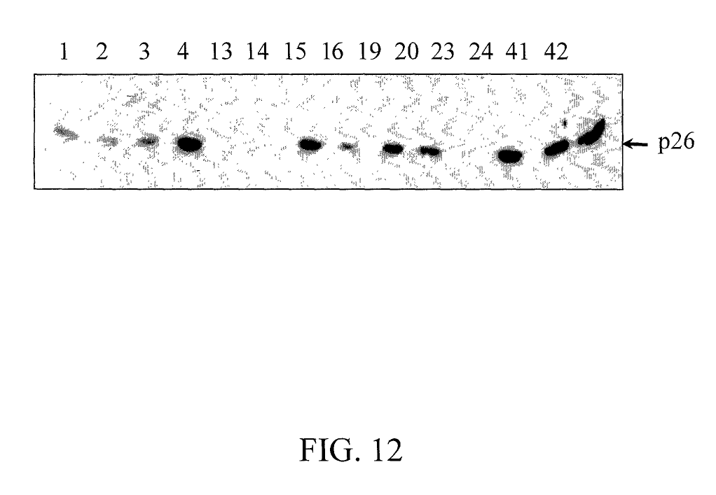

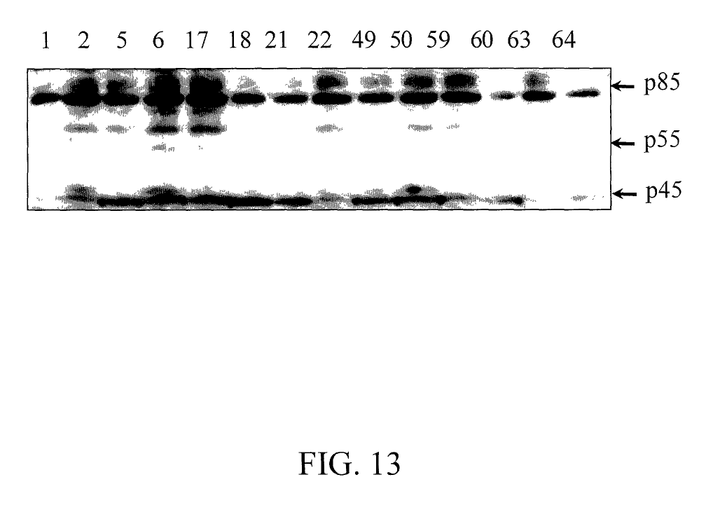

Informative markers for detection of modified protein complexes are detected by the GSTFyn-SH2 in combination with anti-phosphotyrosine antibodies. After affinity purification on GST-Fyn SH2 of the cellular proteins, the bound proteins, constituting a part of the MPCs, are run on SDS-PAGE, and immunoblotted with anti-phosphotyrosine antibodies. Seven cancer patients are shown in FIG. 11, 12 and 13, with the cancer biopsies in even lanes and the corresponding non-cancer biopsies in odd lanes. FIG. 11 shows the 52 KD phosphoprotein, while FIG. 12 shows the 26 KD phosphoprotein. FIG. 13 shows the results obtained by releasing anti-p85 reactive proteins bound to GST-Fyn SH2 protein. After affinity purification on GST-Fyn SH2 of the cellular proteins, the bound proteins, constituting a part of the MPCs, are run on SDS-PAGE, and immunoblotted with anti-p85 antibodies. Seven cancer patients are evaluated, with the cancer biopsies in even lanes, and the corresponding non-cancer biopsies in odd lanes. An anti-p85 reactive proteins (i.e. the 45 kd) is present only in cancer biopsies, and not in normal tissues from the same patients, while for example the p55 form is present in both cancer and normal tissues from a patient. This activation of the p55 from present in normal tissue could represent either a genetic predisposition of the patient or a pre-cancerous lesion, since it is not present in a number of other patients, and in both cases it can be of precious diagnostical meaning.

### EXAMPLE 8

### Grb2 binds to a pTyr Containing Modified Protein Complexes in Cancer

Materials and Methods

In vitro binding studies using GST-Grb2 fusion protein (Bardelli A, Basile M L, Audero E, Giordano S, Wennstrom S, Menard S, Comoglio P M, Ponzetto C Concomitant activation of pathways downstream of Grb2 and PI 3-kinase is required for MET-mediated metastasis. Oncogene 1999 18:1139-46; Cheng A M, Saxton T M, Sakai R, Kulkarni S, Mbamalu G, Vogel W, Tortorice C G, Cardiff RD, Cross J C, Muller W J, Pawson T Mammalian Grb2 regulates multiple steps in embryonic development and malignant transformation. Cell 1998 95:793-803). The whole Grb2 cDNA was isolated using polymerase chain reaction (PCR) and cloned into the BamHI-EcoRI sites of the bacterial expression plasmid pGEX-2T (GST-Grb2). Cultures of bacteria expressing GST, GST-Grb2 were grown for 3-4 hours at 37.degree. Celsius in LB medium containing 1 mM IPTG. Bacteria were centrifuged, resuspended in 1/100 volume of ice-cold PY buffer, without Triton .TM. and lysed by sonication. After adding Triton X-100 to 1%, lysates were clarified by centrifugation. Recombinant proteins were purified onto glutathione Sepharose TM. (Pharmacia) and used as such for binding assays. For each reaction, about 5 .mu.g of GST-Grb2 bound to glutathione Sepharose was incubated for 2 hrs. at 4 .degree. Celsius with 300 mg of appropriate cell lysate made in PY buffer. Protein complexes were washed 5 times in ice cold PY buffer, eluted and denatured by heating at 95.degree. Celsius for 3 min in Laemmli buffer, resolved on SDS-PAGE and analyzed by immunoblot.

Results and Discussion

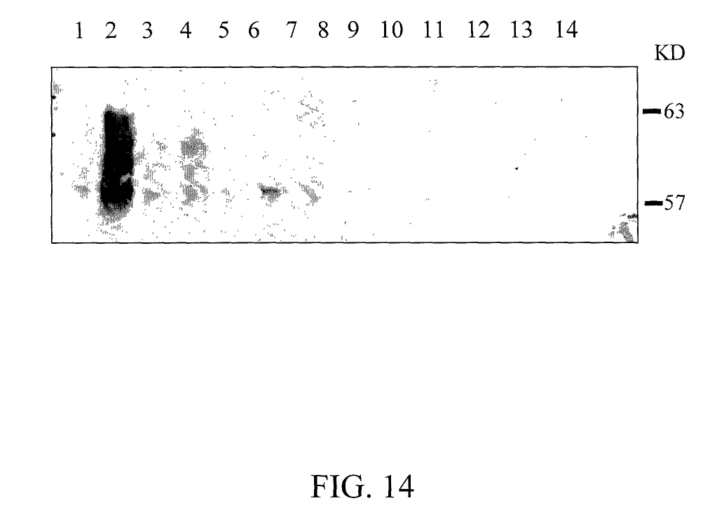

We have screened 22 patients' tumor and normal biopsies, with GST-Grb2. Affinity purified binding proteins were revealed by a set of protein-specific and general context antibodies in western blotting. Informative markers for detection of modified protein complexes are the GST-Grb2 in combination with anti-phosphotyrosine. FIG. 14 shows the results obtained by releasing phosphotyrosine phosphorylated proteins bound to GST-Grb2 protein. After affinity purification on GST-Grb2 of the cellular proteins, the bound proteins, constituting a part of the MPCs, are run on SDS-PAGE, and immunoblotted with anti-phosphotyrosine antibodies. Seven cancer patients are shown, with the cancer biopsies in even lanes, and the corresponding non-cancer biopsies in odd lanes. A range of phosphotyrosine reactive proteins (MW of 57 KD, 60 KD and 63 KD) are present only in cancer biopsies, and not in normal tissues from the same patients.

### EXAMPLE 9

### The Presence of SHC in an Activated Complex with GST-p85

Materials and Methods

In vitro binding studies using GST-p85 fusion protein. The whole p85 alpha human cDNA was isolated using polymerase chain reaction (PCR) and cloned into the EcoRI site of the bacterial expression plasmid pGEX-2T (GST-p85). Cultures of bacteria expressing GST-p85 were grown for 3-4 hours at 37.degree. Celsius in LB medium containing 1 mM IPTG. Bacteria were centrifuged, resuspended in 1/100 volume of ice-cold PY buffer, without Triton TM. and lysed by sonication. After adding Triton X-100 to 1%, lysates were clarified by centrifugation. Recombinant proteins were purified onto glutathione Sepharose .TM. (Pharmacia) and used as such for binding assays. For each reaction, about 5 .mu.g of GST-p85 bound to glutathione Sepharose was incubated for 2 hrs. at 4 degree. Celsius with 300 mg of appropriate cell lysate made in PY buffer. Protein complexes were washed 5 times in ice cold PY buffer, eluted and denatured by heating at 95.degree. Celsius for 3 min in Laemmli buffer, resolved on SDS-PAGE and analyzed by immunoblot.

Results

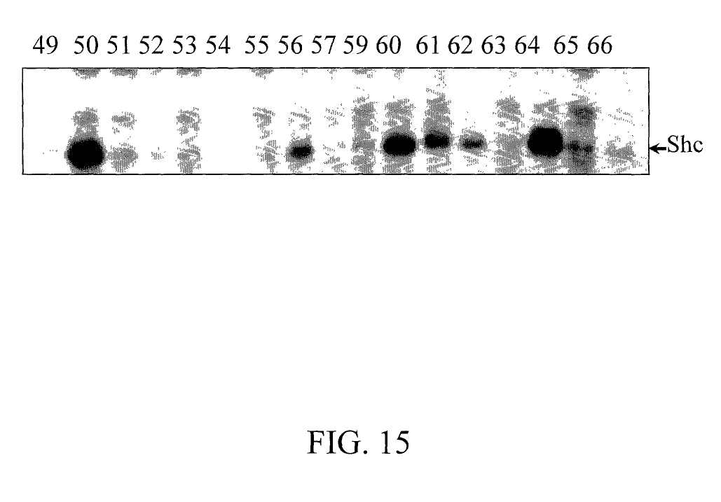

We have found that the p85 subunit of P13 kinase associates in a complex with the proteins Shc preferentially in tumors which have metastatic properties, this interaction is not present, or present at a low level, in non metastatic tumors, and in normal cells. FIG. 15 shows the results obtained by releasing SHC bound to GST-p85 protein. After affinity purification on GST-p85 of the cellular proteins, the bound proteins, constituting a part of the MPCs, are run on SDS-PAGE, and immunoblotted with anti-SHC antibodies. Seven cancer patients are shown, with the cancer biopsies in even lanes, and the corresponding non-cancer biopsies in odd lanes. The SHC reactive protein is present often in cancer biopsies and very rarely in normal tissues from the same patients. Furthermore it positively correlates with metastatic potential.

Discussion

From the above it clearly appears that the presence of a phosphorylated phosphotyrosine complex which includes p85, SHC might be a critical step in the signaling pathway leading to cell tumor invasiveness and ultimately metastasis, at least in head and neck cancer. Furthermore these finding were confirmed in colon cancer.

Since these biological responses constitute the most important characteristics of tumor growth and spreading, need for detection and interfering in such interaction is recognized in the art. A method has been developed for quantitative detection of p85, SHC complex for evaluation of metastatic potential of tumor cells. First, the cells to be examined and evaluated are selected. The cells can be obtained from known tumor cell lines cultured for research purposes, from tumor biopsies or cytological samples from patients or any other source of tissue to be examined for metastatic activity of tumor cells. The cell sample preparations are incubated with immobilized p85 in order to assay for the presence of p85 binding activated complexes. The sample of cells may be prepared in suspension for analysis by analytical cytometry techniques such as flow cytometry, digital image analysis or sectioned and prepared as histology slides for digital image analysis. Then SHC presence in the p85 binding complexes is assayed by Western Blotting or ELISA or other immunochemical techniques, such as energy transfer between fluorescent molecules. The antibodies specific to SHC may be labeled with a fluorescent marker detectable by analytical cytometry techniques and the presence of the complex detected if the p85 is labeled with a different fluorescent marker. Relatively high activated metastatic complexes will indicate the need for aggressive oncology, radiation or immunologic cancer therapy and adjuvant treatment following surgical excision of the tumor. Furthermore p85, SHC complex presence indicates that a combined pharmacological treatment including two different SH2 inhibitors specific for both p85 and SHC should be deployed. By establishing a correlation between p85, SHC complex and metastasis a screening process can be used to identify metastatic tumor cells. The isolated tissue is examined for cells containing p85, SHC complex.

The presence of an activated p85, SHC complex can be due to a number of DNA mutations involving a number of genes present on different chromosomes, and thus can not be easily detected by DNA screening techniques. In fact an activated p85, SHC complex could be the result of mutations in a receptor which activate it constitutively, mutations in a phosphatase which normally down regulates activated receptors, mutations in cytoplasmic kinases which activate non tyrosine kinase receptors, DNA mutations affecting the transcription rate or the conformation and stability of growth factors and many other molecular effectors and enzymes. Thus the p85, SHC complex can be best detected by using biochemical techniques to see the net results of cellular mutations. Antibodies can be formed against the two major molecular forms of p85, SHC complex using established methods of isolating the p85, SHC complex or p85, SHC proteins then immunizing animals to produce the antibodies. Typically, the p85 and SHC antigens are isolated from human cell culture by absorption chromatography. Most commercial anti-p85 or SHC antibodies are murine or rabbit antibodies made against human p85, SHC. Commercial antibodies (such as UBI, SC, etc.) can detect the 46 52 and 66 forms of SHC, but cannot distinguish between the phosphorylated molecular forms, similarly for p85. It is important to be able to detect when p85 and SHC are binding to the same activated phosphoproteins. Specific phospho-antibodies could be made to a host of substrates which, when tyrosine phosphorylated, can bind to p85 and SHC, but it is not possible in this way to determine whether both p85 and SHC are binding to the same activated receptor/adapter. The preferred method is therefore to use immobilized or labeled SH2 domains to discriminate between activated and not activated complexes. Presently, none of the commercial anti-p85, SHC antibodies can detect the presence of such a complex in the cell. Probably due to the size of the activated complexes and the apparent inaccessibility of the antibodies to the native complex. SH2 domains due to their high avidity, fast on-off rate, and high specificity are the method of choice to identify activated p85, SHC complexes. The activated complex is likely to be constituted by one or more bridging molecules which, in order to exert their full metastatic potential, need to recruit both p85 and SHC. Such a molecule can be either a receptor or an adapter, such for example the IRS family. The methods we devised and use does not need to know about which receptor or other bridging molecule is activated, but only that both binding sites for p85 and SHC are phosphorylated. This feature confers to our method a valence, which goes beyond the tissue specific expression of a receptor or other activated molecule, and in fact makes the method a multivalent system for detection of activated metastatic complexes in a number of tissues and tumours. This multivalence is demonstrated in two tumours: colon carcinoma and head and neck tumours.

The cells to be examined are first isolated. The cells may be from a tumor, fine-needle biopsy or cytological sample. The anti-p85, SHC complex antibodies are incubated with the cells. In a preferred method for examination of intracellular p85, SHC complex, the cells are labeled with fluorescent markers for digital image analysis. Using digital image analysis the anti-p85, SHC complex antibodies can be located and quantitatively measured in both the cytoplasm and where the p85, SHC complex is bound to the cell membrane. These measurements are used statistically to give the relative distribution and absolute concentrations of membrane-bound p85/SHC complex in biopsy cells. This data can then be used to statistically compare the levels and distribution in those cells with tumor cells from other patients, thus giving a quantitative benchmark of those tumors in each individual patient. These data also can be used in retrospective studies where the time to reoccurrence, degree of metastasis and morbidity are known. Cumulative data on patients can then be used to provide a prognostic indicator of the degree of active metastasis in primary tumors.

In biopsies from lymph nodes positive patients' p52 Shc proteins is often present in a complex, which binds the p85 subunit of PI 3-kinase (FIG. 15). Only one tumor out of 22 (P<0.05), which is lymph nodes positive, is GSTp85/SHC negative. This patient might have an alternative or most likely downstream-activated effector.

Harrison et al. (Harrison-Findik D, Susa M, Varticovski L Association of phosphatidylinositol 3-kinase with SHC in chronic myelogeneous leukemia cells. Oncogene 1995 10:1385-91) show that PI 3-Kinase directly associates with Shc in hematopoietic cells transformed by BCR/Abl oncoprotein. We show that the interaction is not due to p85 SH3, since the corresponding affinity purification assay was negative. We show in the following example that SHC binds, in the cancer biopsies, to a phosphoprotein complex containing p85 with either its SH2 or its PTB domain.

### EXAMPLE 10

### PTB and SH2 of SHC Bind Differently to Modified Protein Complexes Containing p85 in Tumors

Materials and Methods

In vitro binding studies using GST-SHC PTB and GST-SHC SH2 fusion proteins. The respective cDNAs were isolated using polymerase chain reaction (PCR) and cloned into the BamHI-EcoRI sites of the bacterial expression plasmid pGEX-2T (GST-p85). Cultures of bacteria expressing GST-SHC PTB and GST-SHC SH2 fusion proteins were grown for 3-4 hours at 37.degree. Celsius in LB medium containing 1 mM IPTG. Bacteria were centrifuged, resuspended in 1/100 volume of ice-cold PY buffer, without Triton .TM. and lysed by sonication. After adding Triton X-100 to 1%, lysates were clarified by centrifugation. Recombinant proteins were purified onto glutathione Sepharose .TM. (Pharmacia) and used as such for binding assays. For each reaction, about 5 .mu.g of GST-SHC PTB or GST-SHC SH2 fusion proteins bound to glutathione Sepharose was incubated for 2 hrs. at 4 degree. Celsius with 300 mg of appropriate cell lysate made in PY buffer. Protein complexes were washed 5 times in ice cold PY buffer, eluted and denatured by heating at 95.degree. Celsius for 3 min in Laemmli buffer, resolved on SDS-PAGE and analyzed by immunoblot.

Results

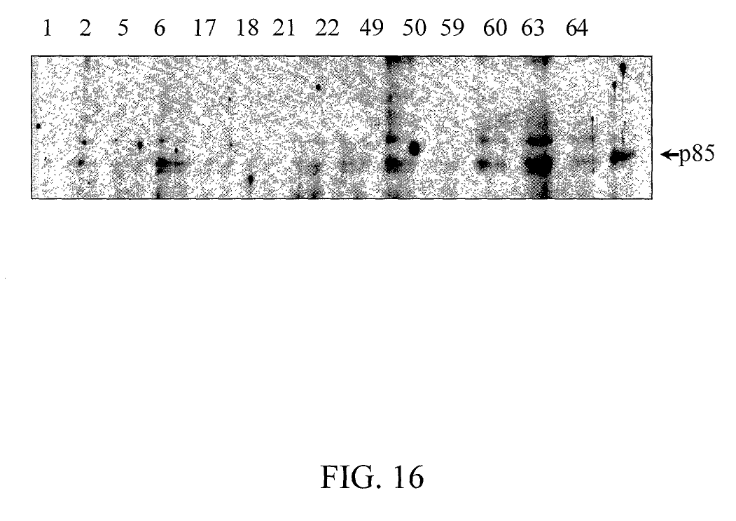

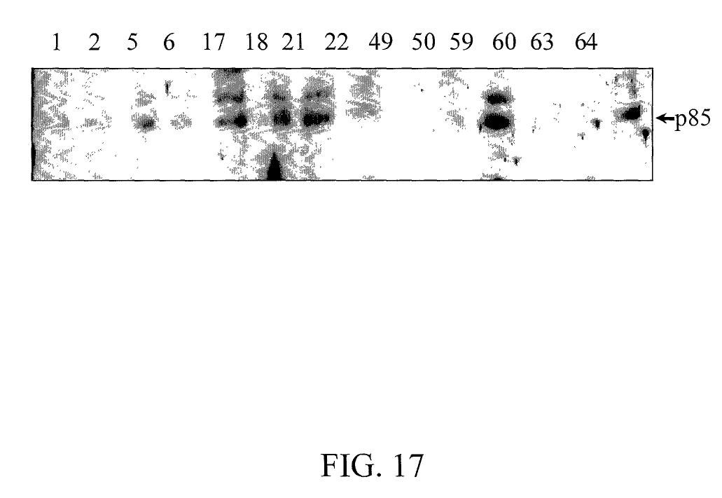

Additional informative markers are the PTB of SHC (FIG. 16) and the SH2 domain of SHC (FIG. 17) when used in combination with p85 detection. These markers differentiate two groups from the GSTp85/SHC positive patients' population. It seems that the presence of a SHC PTB binding site in the p85-associated complex has the ability of blocking the metastatic potential of the activated GST-p85-SHC complex, since positive tumors are lymph nodes negative.

The PTB domain of SHC, a non-metastatic phenotype when in association to PI 3-kinase, might have to bind a different, yet undetermined molecule, in order to be fully metastatic and not the common p85/SHC receptor/adapter, possibly engaged by the SHC SH2 or by some other adapter. We have found that the p85 subunit of P13 kinase associates in a complex with Shc in tumors, which have metastatic properties, but not in non-metastatic tumors (only in less than 5% of the cases), and in normal cells. From the above it appears that the presence of a phosphorylated phosphotyrosine complex which includes p85 and SHC is a step often associated to cell tumor invasiveness and metastasis, particularly when the PTB domain of SHC is not used towards p85 binding.

Discussion

Recently Chin et al (Chin L, Tam A, Pomerantz J, Wong M, Holash J, Bardeesy N, Shen Q, O'Hagan R, Pantginis J, Zhou H, Homer J W 2nd, Cordon-Cardo C, Yancopoulos G D, DePinho RA Nature 1999 400:468-72. Essential role for oncogenic Ras in tumour maintenance), have shown that melanoma maintenance is strictly dependent upon expression of H-RasV12G in an inducible H-Rasl2G mouse melanoma model null for the tumour suppressor INK4a. H-RasV12G down-regulation resulted in clinical and histological regression of primary and explanted tumours. The initial stages of regression involved marked apoptosis in the tumour cells and host-derived endothelial cells. Although the regulation of vascular endothelial growth factor (VEGF) was found to be Ras-dependent in vitro, the failure of persistent endogenous and enforced VEGF expression to sustain tumour viability indicates that the tumour-maintaining actions of activated Ras extend beyond the regulation of VEGF expression in vivo. Our results provide an evidence that Shc activation and recruitment is necessary in spontaneous solid tumours, as long as it is also supported by a PI 3-kinase activation. Any receptor or membrane targeted adapter which can recruit both effectors to the membrane, thereby activating the two downstream pathways, is likely to provoke similar metastatic response. Furthermore p85, SHC complex presence indicates that a combined pharmacological treatment including two different SH2 inhibitors specific for both p85 and SHC should be deployed. A second marker, which can be used to detect metastasis prone tumors, is the above described Fyn-SH2 binding and tyrosine phosphorylated p26 protein.

### EXAMPLE 11

### Pin1 can Bind Differently Phosphothreonine- and Phosphoserine-containing MPCs in Cancer Biopsies

Materials and Methods

Cloning and expression of human Pin1. RT-PCR was used in order to clone Pin1 in BamHI/EcoRI cleaved pGEX2TK vector (Pharmacia), by using turbo-Pfu DNA polymerase (Stratagene) and human first strand cDNA from a range of human cell lines. Oligonucleotides used for Pin1 sense CAGGGATCCATGGCGGACGAGGAGAAGC, antisense GACGAATTCTCACTCAGTGCGGAGGATG. The clones were sequenced to confirm their identity. The bacterially expressed fusion proteins were purified on Glutathione-Sepharose (Pharmacia).

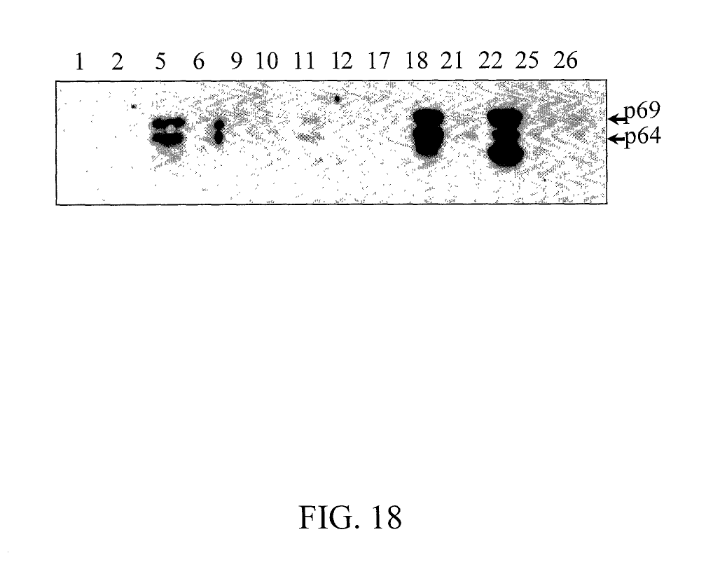

GST-Pin1 was also used in combination with anti-phosphothreonine antibody, when a phosphorylated doublet of about 64 and 69 Kd was observed in metastatic tumors. FIG. 18 shows the results obtained by releasing phosphotyrosine phosphorylated proteins bound to GST-Pin1 protein. After affinity purification on GST-Pin1 of the cellular proteins, the bound proteins are run on SDS-PAGE, and immunoblotted with anti-phosphothreonine antibodies. 22 cancer patients are evaluated, with the cancer biopsies in even lanes, and the corresponding non-cancer biopsies in odd lanes. Threonine phosphorylated proteins, such as the p64/p69 doublet, when bound to Pin1 have a good correlation with lymph node positive tumor.

### EXAMPLE 12

### Pin1 can Bind Differently pTyr MPCs in Cancer Biopsies

Results and Discussion

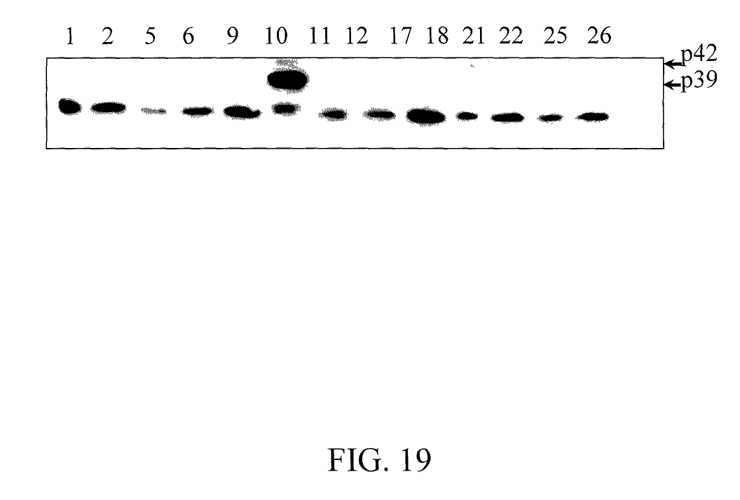

We also investigated the combined involvement of serine/threonine and tyrosine phosphorylation in cancer. To detect this combination we used the phosphoserine/threonine dependent proline isomerase Pin1 in a fusion protein with GST, as a phosphorylation trap and immunoblotting with anti-phosphotyrosine. This marker can detect a cancer state in the normal tissues, where the effect of a pre-cancer mutation or of growth factors secreted from the tumour cells, are measured. FIG. 19 shows the results obtained by releasing phosphotyrosine phosphorylated proteins bound to GST-Pin1 protein. After affinity purification on GST-Pin1 of the cellular proteins, the bound proteins are run on SDS-PAGE, and immunoblotted with anti-anti-phosphotyrosine antibodies. Seven cancer patients are shown, with the cancer biopsies in even lanes, and the corresponding non-cancer biopsies in odd lanes. p39 and p42 are abnormal proteins detected in cancer patients.

### EXAMPLE 13

### 14-3-3 Binds Differentially Phosphothreonine-containing MPCs in Metastatic Cancer Biopsies

Materials and Methods

Cloning and expression of human 14-3-3 epsilon. RT-PCR was used in order to clone 14-3-3 in BamHI/EcoRI cleaved pGEX2TK vector (Pharmacia), by using turbo-Pfu DNA polymerase (Stratagene) and human first strand cDNA from a range of human cell lines. Oligonucleotides used for 14-3-3 epsilon sense CCGGATCCATGGATGATCGAGAGGATCTGGTG, antisense GGAATCCTCACTGATTTTCGTCTTCCACGTCC. The clones were sequenced to confirm their identity. The bacterially expressed fusion proteins were purified on Glutathione-Sepharose (Pharmacia).

Results and Discussion

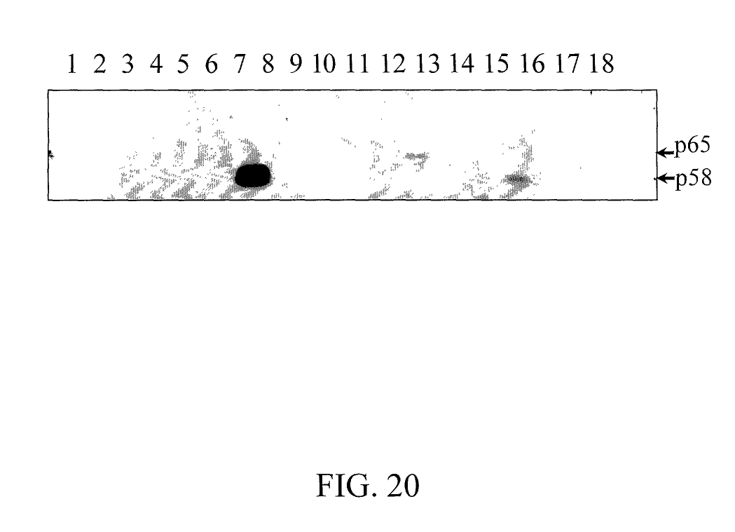

GST-14-3-3 epsilon was also used in combination with anti-phosphothreonine antibody to reveal a phosphorylated form of about 65 Kd in metastatic tumors. FIG. 20 shows the results obtained by releasing phosphothreonine proteins bound to GST-14-3-3 epsilon. After affinity purification on GST-14-3-3 epsilon of the cellular proteins, the bound proteins are run on SDS-PAGE, and immunoblotted with anti-phosphothreonine antibodies. Nine cancer patients are evaluated, with the cancer biopsies in even lanes, and the corresponding non-cancer biopsies in odd lanes.

### EXAMPLE 14

### Algorithms can Differentiate Tumor Biopsies Based on Molecular Profiling of Modified Protein Complexes

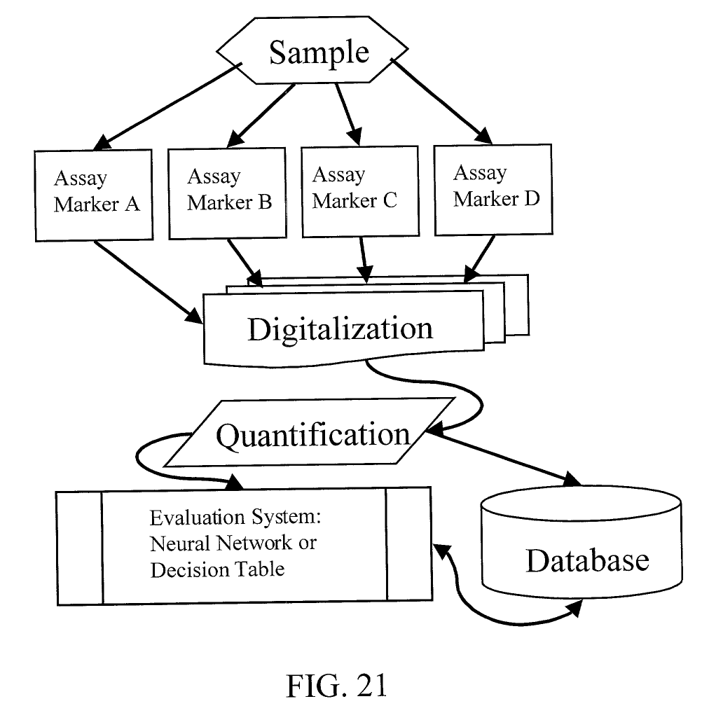

By using data mining algorithms we have explored the association between these markers and cancer and metastasis, as illustrated in FIG. 21. Classifier IB1 and the two markers GST-p85/SHC Fyn/p26 enable 100% prediction of lymph node positives and negatives (IB1 instance-based classifier using 1 nearest neighbor(s) for classification with 8% of relative error).

Materials and Methods

Digitalization. The western blots were digitized and analysed with Scion Image (Scion Corporation).

Statistical Analysis. To classify the variables in relation to the class lymph nodes the package WEKA (Witten I. H. and Frank E. (2000) Morgan Kaufmann, San Francisco) was used.

Results and Discussion

The combination of the use of these markers enables the evaluation of cancer prognosis and metastatic potential in a cancer biopsy. Genetic background differences in different patients might be evident by studying molecular profiling patterns, and it is evident in our results that also the difference between the “normal” and cancer biopsies from the same patients is a key to a successful molecular diagnosis. In fact the ratio between the non-pathological and pathological value of a marker is also used in our analysis, alongside absolute values. Furthermore pre-cancerous states and/or activated states in normal tissues, possibly revealing a tumour with paracrine activity, in a different location from that of the biopsy, could be detected by this phosphorylation analysis, which could thus be applied to early detection and diagnosis of neoplastic alterations.

By interfering with signal transduction mechanisms, often growth and diffusion of tumour cells can be inhibited: for example, inhibiting MEK, and therefore blocking MAPKs, in vivo growth of colon cancer cells is suppressed (Sebolt-Leopold J S, Dudley D T, Herrera R, Van Becelaere K, Wiland A, Gowan R C, Tecle H, Barrett S D, Bridges A, Przybranowski S, Leopold W R, Saltiel A R Nat Med 1999 Jul; 5(7):810-6 Blockade of the MAP kinase pathway suppresses growth of colon tumors in vivo.) or modulation of androgen receptor response by HER-2/neu tyrosine kinase could be a mechanism contributing to onset of prostate cancer (Craft N, Shostak Y, Carey M & Sawyers CL “A mechanism for hormone-independent prostate cancer through modulation of androgen receptor signaling by the HER-2/neu tyrosine kinase” Nat Med 5:280-5 1999). Furthermore, even when not directly involved in the cell growth, tyrosine kinase receptors are important for tumour growth. Vascular endothelial growth factor receptor VEGFR-2 is linked to angiogenesis and neoplasm invasiveness (Skobe M, Rockwell P, Goldstein N, Vosseler S, Fusenig N E Nat Med 1997 11:1222-7 Halting angiogenesis suppresses carcinoma cell invasion.). Selective inhibitors of tyrosine kinases are under investigation for tumour treatment, like in the case of chronic myeloid leukemia (CML), where Bcr-Abl fusion protein is present in 95% of patients (Druker B J, Tamura S, Buchdunger E, Ohno S, Segal G M, Fanning S, Zimmermann J, Lydon N B Nat Med 1996 5:561-6 Effects of a selective inhibitor of the Abl tyrosine kinase on the growth of Bcr-Abl positive cells.). Activated receptor inhibition, by using immuno-therapy, has been obtained in mice, where it has been possible to prevent breast cancer by injecting p185^{neu} specific monoclonal antibodies (Katsumata et al, 1995).

### EXAMPLE 15

### Peptides for Modulation of Metastatic Specific Alterations Detected in Previous Examples

Materials and Methods

Peptides. Phosphotyrosine-containing peptides were synthesized, HPLC purified and checked by mass spectroscopy. Peptides were dissolved in 50 mM NaPO buffer, pH 6.5, and stored under nitrogen at −80 ° C.

Results and Discussion

After identification of specific modified complexes correlating with metastasis in cancer, we have designed phosphopeptides in order to compete and block metastasis. In the case of the p85/SHC interaction two peptides have been designed: (1) a p85 SH2 binding phosphopeptide coupled with a SHC SH2 binding phosphopeptide through a spacer, DDGpYMPMS-spacer-GpYIGI and (2) a p85 SH2 binding phosphopeptide coupled with a SHC PTB binding phosphopeptide through a spacer, DDGpYMPMS-spacer-FGNPIpYG. In the case of the Fyn/pTyr interaction a peptide has been designed: a Fyn SH2 binding phosphopeptide coupled with a Grb2 phosphopeptide through a spacer QpYEEI-spacer-GpYQNQ. In the case of the Pin1 phosphoprotein: (1) a Pin1 binding phosphopeptide, SpSPGpSPGpTPGSRSRpTPSLPpTPPTRE. In the case of the 14-3-3 phosphoprotein: (1) a 14-3-3 binding phosphopeptide, RLYHpSLP.

Having now described a few embodiments, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention.

What is claimed is:

1. A method of analyzing the activity or level of one or more protein or enzyme, said method comprising:
(a) providing a pool of substrates (peptides, antibodies, binding domains, other molecules acting as substrates or control substrates) each with a specific tag and representing a substrate of one or more of said proteins or enzymes, or substrates derived therefrom using said tagged substrates as substrates;
(b) hybridizing said pool of tagged substrates to an ordered array of specific and complementary tags immobilized on a surface, said array comprising more different tags, at least some of which comprise control tags, wherein each tag is localized in a predetermined region of said surface, the density of said different tags is greater than about 100 different tags per 1 cm.sup.2, and all tags in the substrates derived therefrom using said proteins or enzymes are complementary to at least some of the immobilized tags;
(c) quantifying the hybridization of said substrates tagged with nucleic acids or PNAs to said array, wherein said quantification is proportional to the activity of proteins or enzymes modifying or attaching to the substrates tagged with nucleic acids or PNAs.

2. The method of claim 1, wherein said pool of substrates each tagged with a single tag comprises substrates tagged with nucleic acids or PNAs and wherein said ordered array of specific and complementary tags immobilized on a surface comprises ordered array of specific and complementary nucleic acids or PNAs immobilized on a surface .

3. The method of claim 2, wherein said quantifying comprises calculating the difference in hybridization signal intensity between each of said substrates tagged with a single nucleic acid or PNA and its corresponding related elements.

4. The method of claim 3, wherein said quantifying comprises calculating the average difference in hybridization signal intensity between each of said substrates tagged with a single nucleic acid or PNA and its corresponding control substrate for each protein or enzyme, where the control substrate has either an identical tag or a different tag.

5. The method of claim 1, wherein said multiplicity of substrates tagged with a nucleic acids or PNAs is 100 or more.

6. The method of claim 1, wherein for each said protein or enzyme, said array comprises at least 8 different substrates tagged with a nucleic acids or PNAs acting as substrates.

7. The method of claim 1, wherein said hybridization is performed with a fluid volume of about 200 .mu.l or less.

8. The method of claim 1, wherein said quantifying comprises detecting a hybridization signal that is proportional to the concentration of modified substrates tagged with a nucleic acids or PNAs in said tagged substrates pool.

9. The method of claim 1, wherein said substrates nucleic acid or PNA tags are at least 21 nucleotides in length.

10. The method of claim 1, wherein said control substrates comprise either premodified substrates or substrates which are substrates of constitutively expressed control proteins or enzymes.

11. The method of claim 10, wherein said tagged substrates include GST-Pin1, GST-14-3-3, GSTFyn SH2, GST-p85, GST-Shc PTB, GST-Shc SH2 and GST-Grb2, and said control substrates are selected from the group consisting of substrates for protein kinase C alpha., protein kinase C .beta.1 , protein kinase C .beta.2, protein kinase C .gamma. phosphatidylinositol 3-kinase alpha., phosphatidylinositol 3-kinase beta., phosphatidylinositol 3-kinase C2 .beta., phosphatidylinositol 3-kinase C2 gamma, src, abl, PDGF receptor.

12. The method of claim 1, wherein said hybridization comprises a hybridization at low stringency of 42.degree. C. to 54.degree. C. and 3× TBST and a wash at higher stringency.

13. The method of claim 1, wherein said pool of substrates each tagged with a single nucleic acid or PNA comprises fluorescently labeled substrates.

14. The method of claim 1, wherein said quantifying comprises quantifying fluorescence of a label on said hybridized tagged substrate at a spatial resolution of about 100 .mu.m or higher.

15. The method of claim 1, wherein said providing comprises:
(i) treating said pool of tagged substrates with protein or enzyme samples, thereby modifying the tagged substrates and leaving intact the tag single stranded component of each tagged substrate;
(ii) isolating the tagged substrates pool thereby leaving a pool of substrates modified by those protein or enzymes present and active in the protein or enzyme sample.

16. A method of analyzing the activity of one or more protein or enzyme, said method comprising:
(a) providing a pool of molecules (peptides, antibodies, binding domains, other molecules acting as substrates or control substrates) and representing a substrate of one or more of said proteins or enzymes, or substrates derived therefrom;
(b) reacting said pool of molecules to an array of proteins, peptides, or other non DNA molecules, immobilized on a surface, wherein each different protein, peptide, or other non DNA molecule is localized in a predetermined region of said surface, the density of said different proteins, peptides, or other molecules, is greater than about 60 different oligonucleotides per 1 cm.sup.2,;
(c) quantifying the reactivity of said array, wherein said quantification is proportional to the activity of proteins or enzymes modifying or attaching to the substrates tagged with nucleic acids or PNAs.

17. The method of claim 16, wherein said pool of molecules further comprises the same substrate for more than one different element in the said array.

18. The method of claim 17, wherein said quantifying comprises calculating the difference in signal intensity between each of said array elements.

19. The method of claim 18, wherein said quantifying comprises calculating the average difference in signal intensity between each of said array element and its corresponding control substrate for each protein or enzyme.

20. The method of claim 16, wherein said multiplicity of array elements is 100 or more.

21. The method of claim 16, wherein for each said protein or enzyme, said array comprises at least different reactive elements.

22. The method of claim 16, wherein said hybridization is performed with a fluid volume of about 200 .mu.l or less.

23. The method of claim 16, wherein said quantifying comprises detecting a hybridization signal that is proportional to reacted array element.

24. The method of claim 16, wherein said control substrates comprise either premodified substrates or substrates which are substrates of constitutively expressed control proteins or enzymes.

25. The method of claim 24, wherein said tagged substrates include GST-Pin1, GST-14-3-3, GST-Fyn SH2, GST-p85, GST-Shc PTB, GST-Shc SH2 and GST-Grb2, and said control substrates are selected from the group consisting of substrates for protein kinase C alpha., protein kinase C beta. 1 , protein kinase C .beta.2, protein kinase C gamma. phosphatidylinositol 3-kinase .alpha., phosphatidylinositol 3-kinase .beta., phosphatidylinositol 3-kinase C2 beta., phosphatidylinositol 3-kinase C2 gamma, src, abl, PDGF receptor.

26. The method of claim 16, wherein said reacting comprises a reaction at 4.degree. C. to 37.degree. C. and 1.5× TBST and a wash at 2xTBST.

27. The method of claim 16, wherein said pool of molecules comprises fluorescently labeled molecules.

28. The method of claim 16, wherein said quantifying comprises quantifying fluorescence of a label on said reacted substrate at a spatial resolution of about 100 .mu.m or higher.
